SEQUENCE LISTING (152 sequences)

SEQ ID NO: 1 - protein, 64 aa, Artificial Sequence
  Synthetic peptide
GDVNDDGKVNSTDAVALKRYVLRSGISINTDNADLNEDGRVNSTDLGILKRYILKEIDTLPYKN

SEQ ID NO: 2 - protein, 48 aa, Artificial Sequence
  Synthetic peptide
GKRYVLRSGISINTDNADLNEDGRVNSTDLGILKRYILKEIDTLPYKN

SEQ ID NO: 3 - protein, 41 aa, Artificial Sequence
  Synthetic peptide
GKRYVLRSGISINTDNADLNEDGRVNSTDLGILKRYILKEI

SEQ ID NO: 4 - protein, 48 aa, Artificial Sequence
  Synthetic peptide
GDVNDDGKVNSTDAVALKRYVLRSGISINTDNAKRYILKEIDTLPYKN

SEQ ID NO: 5 - protein, 47 aa, Artificial Sequence
  Synthetic peptide
KRYVLRSGISINTDNADLNEDGRVNSTDLGILKRYILKEIDTLPYKN

SEQ ID NO: 6 - protein, 50 aa, Artificial Sequence
  Synthetic peptide
MGSKRYVLRSGISINTDNADLNEDGRVNSTDLGILKRYILKEIDTLPYKN

SEQ ID NO: 7 - protein, 741 aa, Clostridium thermocellum
MVKSRKISILLAVAMLVSIMIPTTAFAGPTKAPTKDGTSYKDLFLELYGKIKDPKNGYFSPDEGIPYHSIETLIVEAPDYGHVTTSEAFSYYVWLEAMYGNLTGNWSGVETAWKVMEDWIIPDSTEQPGMSSYNPNSPATYADEYEDPSYYPSELKFDTVRVGSDPVHNDLVSAYGPNMYLMHWLMDVDNWYGFGTGTRATFINTFQRGEQESTWETIPHPSIEEFKYGGPNGFLDLFTKDRSYAKQWRYTNAPDAEGRAIQAVYWANKWAKEQGKGSAVASVVSKAAKMGDFLRNDMFDKYFMKIGAQDKTPATGYDSAHYLMAWYTAWGGGIGASWAWKIGCSHAHFGYQNPFQGWVSATQSDFAPKSSNGKRDWTTSYKRQLEFYQWLQSAEGGIAGGATNSWNGRYEKYPAGTSTFYGMAYVPHPVYADPGSNQWFGFQAWSMQRVMEYYLETGDSSVKNLIKKWVDWVMSEIKLYDDGTFAIPSDLEWSGQPDTWTGTYTGNPNLHVRVTSYGTDLGVAGSLANALATYAAATERWEGKLDTKARDMAAELVNRAWYNFYCSEGKGVVTEEARADYKRFFEQEVYVPAGWSGTMPNGDKIQPGIKFIDIRTKYRQDPYYDIVYQAYLRGEAPVLNYHRFWHEVDLAVAMGVLATYFPDMTYKVPGTPSTKLYGDVNDDGKVNSTDAVALKRYVLRSGISINTDNADLNEDGRVNSTDLGILKRYILKEIDTLPYKN

SEQ ID NO: 8 - protein, 1853 aa, Clostridium thermocellum
MRKVISMLLVVAMLTTIFAAMIPQTVSAATMTVEIGKVTAAVGSKVEIPITLKGVPSKGMANCDFVLGYDPNVLEVTEVKPGSIIKDPDPSKSFDSAIYPDRKMIVFLFAEDSGRGTYAITQDGVFATIVATVKSAAAAPITLLEVGAFADNDLVEISTTFVAGGVNLGSSVPTTQPNVPSDGVVVEIGKVTGSVGTTVEIPVYFRGVPSKGIANCDFVFRYDPNVLEIIGIDPGDIIVDPNPTKSFDTAIYPDRKIIVFLFAEDSGTGAYAITKDGVFAKIRATVKSSAPGYITFDEVGGFADNDLVEQKVSFIDGGVNVGNATPTKGATPTNTATPTKSATATPTRPSVPTNTPTNTPANTPVSGNLKVEFYNSNPSDTTNSINPQFKVTNTGSSAIDLSKLTLRYYYTVDGQKDQTFWCDHAAIIGSNGSYNGITSNVKGTFVKMSSSTNNADTYLEISFTGGTLEPGAHVQIQGRFAKNDWSNYTQSNDYSFKSASQFVEWDQVTAYLNGVLVWGKEPGGSVVPSTQPVTTPPATTKPPATTKPPATTIPPSDDPNAIKIKVDTVNAKPGDTVNIPVRFSGIPSKGIANCDFVYSYDPNVLEIIEIKPGELIVDPNPDKSFDTAVYPDRKIIVFLFAEDSGTGAYAITKDGVFATIVAKVKSGAPNGLSVIKFVEVGGFANNDLVEQRTQFFDGGVNVGDTTVPTTPTTPVTTPTDDSNAVRIKVDTVNAKPGDTVRIPVRFSGIPSKGIANCDFVYSYDPNVLEIIEIEPGDIIVDPNPDKSFDTAVYPDRKIIVFLFAEDSGTGAYAITKDGVFATIVAKVKSGAPNGLSVIKFVEVGGFANNDLVEQKTQFFDGGVNVGDTTEPATPTTPVTTPTTTDDLDAVRIKVDTVNAKPGDTVRIPVRFSGIPSKGIANCDFVYSYDPNVLEIIEIEPGDIIVDPNPDKSFDTAVYPDRKIIVFLFAEDSGTGAYAITKDGVFATIVAKVKSGAPNGLSVIKFVEVGGFANNDLVEQKTQFFDGGVNVGDTTEPATPTTPVTTPTTTDDLDAVRIKVDTVNAKPGDTVRIPVRFSGIPSKGIANCDFVYSYDPNVLEIIEIEPGDIIVDPNPDKSFDTAVYPDRKIIVFLFAEDSGTGAYAITKDGVFATIVAKVKEGAPNGLSVIKFVEVGGFANNDLVEQKTQFFDGGVNVGDTTEPATPTTPVTTPTTTDDLDAVRIKVDTVNAKPGDTVRIPVRFSGIPSKGIANCDFVYSYDPNVLEIIEIEPGELIVDPNPTKSFDTAVYPDRKMIVFLFAEDSGTGAYAITEDGVFATIVAKVKSGAPNGLSVIKFVEVGGFANNDLVEQKTQFFDGGVNVGDTTEPATPTTPVTTPTTTDDLDAVRIKVDTVNAKPGDTVRIPVRFSGIPSKGIANCDFVYSYDPNVLEIIEIEPGDIIVDPNPDKSFDTAVYPDRKIIVFLFAEDSGTGAYAITKDGVFATIVAKVKEGAPNGLSVIKFVEVGGFANNDLVEQKTQFFDGGVNVGDTTVPTTSPTTTPPEPTITPNKLTLKIGRAEGRPGDTVEIPVNLYGVPQKGIASGDFVVSYDPNVLEIIEIEPGELIVDPNPTKSFDTAVYPDRKMIVFLFAEDSGTGAYAITEDGVFATIVAKVKEGAPEGFSAIEISEFGAFADNDLVEVETDLINGGVLVTNKPVIEGYKVSGYILPDFSFDATVAPLVKAGFKVEIVGTELYAVTDANGYFEITGVPANASGYTLKISRATYLDRVIANVVVTGDTSVSTSQAPIMMWVGDIVKDNSINLLDVAEVIRCFNATKGSANYVEELDINRNGAINMQDIMIVHKHFGATSSDYDAQ

SEQ ID NO: 9 - DNA, 5573 bp, Clostridium thermocellum
atgagaaaagtcatcagtatgctcttagttgtggctatgctgacgacgatttttgcggcgatgataccgcagacagtatcggcggccacaatgacagtcgagatcggcaaagttacagcagccgttggatcaaaagtagaaatacctataaccctgaaaggagtgccatccaaaggaatggccaattgcgacttcgtattgggttatgatccaaatgtgctggaagtaacagaagtaaaaccaggaagcataataaaagatccggatcctagcaagagctttgatagcgcaatatatccggatcgaaagatgattgtatttctgtttgcagaagacagtggaagaggaacgtatgcaataactcaggatggagtatttgcaacaattgtagccactgtcaaatcagctgcagcggcaccgattactttgcttgaagtaggtgcatttgcggacaacgatttagtagaaataagcacaacttttgtcgcgggcggagtaaatcttggtagttccgtaccgacaacacagccaaatgttccgtcagacggtgtggtagtagaaattggcaaagttacgggatctgttggaactacagttgaaatacctgtatatttcagaggagttccatccaaaggaatagcaaactgcgactttgtgttcagatatgatccgaatgtattggaaattatagggatagatcccggagacataatagttgacccgaatcctaccaagagctttgatactgcaatatatcctgacagaaagataatagtattcctgtttgcggaagacagcggaacaggagcgtatgcaataactaaagacggagtatttgcaaaaataagagcaactgtaaaatcaagtgctccgggctatattactttcgacgaagtaggtggatttgcagataatgacctggtagaacagaaggtatcatttatagacggtggtgttaacgttggcaatgcaacaccgaccaagggagcaacaccaacaaatacagctacgccgacaaaatcagctacggctacgcccaccaggccatcggtaccgacaaacacaccgacaaacacaccggcaaatacaccggtatcaggcaatttgaaggttgaattctacaacagcaatccttcagatactactaactcaatcaatcctcagttcaaggttactaataccggaagcagtgcaattgatttgtccaaactcacattgagatattattatacagtagacggacagaaagatcagaccttctggtgtgaccatgctgcaataatcggcagtaacggcagctacaacggaattacttcaaatgtaaaaggaacatttgtaaaaatgagttcctcaacaaataacgcagacacctaccttgaaataagctttacaggcggaactcttgaaccgggtgcacatgttcagatacaaggtagatttgcaaagaatgactggagtaactatacacagtcaaatgactactcattcaagtctgcttcacagtttgttgaatgggatcaggtaacagcatacttgaacggtgttcttgtatggggtaaagaacccggtggcagtgtagtaccatcaacacagcctgtaacaacaccacctgcaacaacaaaaccacctgcaacaacaaaaccacctgcaacaacaataccgccgtcagatgatccgaatgcaataaagattaaggtggacacagtaaatgcaaaaccgggagacacagtaaatatacctgtaagattcagtggtataccatccaagggaatagcaaactgtgactttgtatacagctatgacccgaatgtacttgagataatagagataaaaccgggagaattgatagttgacccgaatcctgacaagagctttgatactgcagtatatcctgacagaaagataatagtattcctgtttgcagaagacagcggaacaggagcgtatgcaataactaaagacggagtatttgctacgatagtagcgaaagtaaaatccggagcacctaacggactcagtgtaatcaaatttgtagaagtaggcggatttgcgaacaatgaccttgtagaacagaggacacagttctttgacggtggagtaaatgttggagatacaacagtacctacaacacctacaacacctgtaacaacaccgacagatgattcgaatgcagtaaggattaaggtggacacagtaaatgcaaaaccgggagacacagtaagaatacctgtaagattcagcggtataccatccaagggaatagcaaactgtgactttgtatacagctatgacccgaatgtacttgagataatagagatagaaccgggagacataatagttgacccgaatcctgacaagagctttgatactgcagtatatcctgacagaaagataatagtattcctgtttgcggaagacagcggaacaggagcgtatgcaataactaaagacggagtatttgctacgatagtagcgaaagtaaaatccggagcacctaacggactcagtgtaatcaaatttgtagaagtaggcggatttgcgaacaatgaccttgtagaacagaagacacagttctttgacggtggagtaaatgttggagatacaacagaacctgcaacacctacaacacctgtaacaacaccgacaacaacagatgatctggatgcagtaaggattaaagtggacacagtaaatgcaaaaccgggagacacagtaagaatacctgtaagattcagcggtataccatccaagggaatagcaaactgtgactttgtatacagctatgacccgaatgtacttgagataatagagatagaaccgggagacataatagttgacccgaatcctgacaagagctttgatactgcagtatatcctgacagaaagataatagtattcctgtttgcggaagacagcggaacaggagcgtatgcaataactaaagacggagtatttgctacgatagtagcgaaagtaaaatccggagcacctaacggactcagtgtaatcaaatttgtagaagtaggcggatttgcgaacaatgaccttgtagaacagaagacacagttctttgacggtggagtaaatgttggagatacaacagaacctgcaacacctacaacacctgtaacaacaccgacaacaacagatgatctggatgcagtaaggattaaagtggacacagtaaatgcaaaaccgggagacacagtaagaatacctgtaagattcagcggtataccatccaagggaatagcaaactgtgactttgtatacagctatgacccgaatgtacttgagataatagagatagaaccgggagacataatagttgacccgaatcctgacaagagctttgatactgcagtatatcctgacagaaagataatagtattcctgtttgcagaagacagcggaacaggagcgtatgcaataactaaagacggagtatttgctacgatagtagcgaaagtaaaagaaggagcacctaacggactcagtgtaatcaaatttgtagaagtaggcggatttgcgaacaatgaccttgtagaacagaagacacagttctttgacggtggagtaaatgttggagatacaacagaacctgcaacacctacaacacctgtaacaacaccgacaacaacagatgatctggatgcagtaaggattaaagtggacacagtaaatgcaaaaccgggagacacagtaagaatacctgtaagattcagcggtataccatccaagggaatagcaaactgtgactttgtatacagctatgacccgaatgtacttgagataatagagatagaaccgggagaattgatagttgacccgaatcctaccaagagctttgatactgcagtatatcctgacagaaagatgatagtattcctgtttgcggaagacagcggaacaggagcgtatgcaataactgaagatggagtatttgctacgatagtagcgaaagtaaaatccggagcacctaacggactcagtgtaatcaaatttgtagaagtaggcggatttgcgaacaatgaccttgtagaacagaagacacagttctttgacggtggagtaaatgttggagatacaacagaacctgcaacacctacaacacctgtaacaacaccgacaacaacagatgatctggatgcagtaaggattaaagtggacacagtaaatgcaaaaccgggagacacagtaagaatacctgtaagattcagcggtataccatccaagggaatagcaaactgtgactttgtatacagctatgacccgaatgtacttgagataatagagatagaaccgggagacataatagttgacccgaatcctgacaagagctttgatactgcagtatatcctgacagaaagataatagtattcctgtttgcagaagacagcggaacgggagcgtatgcaataactaaagacggagtatttgctacgatagtagcgaaagtaaaagaaggagcacctaacggactcagtgtaatcaaatttgtagaagtaggcggatttgcgaacaatgaccttgtagaacagaagacacagttctttgacggtggagtaaatgttggagatacaacagtacctacaacatcgccgacaacaacaccgccagagccgacgataactccgaacaagttgacacttaagataggcagagcagaaggaagacctggagacacggtggaaataccggttaacttgtatggagtacctcaaaaaggaatagcaagcggtgacttcgtagtaagctatgacccgaatgtacttgagataatagagatagaaccgggagaattgatagttgacccgaatcctaccaagagctttgatactgctgcagtatatcctgacagaaagatgatagtattcctgtttgcggaagacagcggaacaggagcgtatgcaataactgaagatggagtatttgctacgatagtagcgaaagtaaaagaaggagcacctgaaggattcagtgcaatagaaatttctgagtttggtgcatttgcagataatgatctggtagaagtggaaactgaccttatcaatggtggagtacttgtaactaataaacctgtaatagaaggatataaagtatccggatacattttgccagacttctccttcgacgctactgttgcaccacttgtaaaggccggattcaaagttgaaatagtaggaacagaattgtatgcagtaacagatgcaaacggatactttgaaataaccggagtacctgcaaatgcaagcggatatacattgaagatttcaagagcaacttacttggacagagtaattgcaaatgttgtagtaacgggagatacttcagtttcaacttcacaggctccaataatgatgtgggtaggagacatagtgaaagacaattctatcaacctgttggacgttgcagaagttatccgttgcttcaacgctactaaaggaagcgcaaactacgtagaagaacttgacattaatagaaacggcgcaattaacatgcaagacataatgattgttcataagcactttggagctacatcaagtgattacgacgcacagtaaatattaaa

SEQ ID NO: 10 - protein, 39 aa, Artificial Sequence
  Synthetic peptide
IEFGDVDGNGMIDALDYSLVRKYLLGQISDCPDSKGKLA

SEQ ID NO: 11 - protein, 36 aa, Artificial Sequence
  Synthetic peptide
GLKGDVNNDGAIDALDIAALKKAILTQSTSNINLTN

SEQ ID NO: 12 - protein, 35 aa, Artificial Sequence
  Synthetic peptide
SNLGDVNGDETVDAIDLAMLKKYLLNSSTSIVAGN

SEQ ID NO: 13 - protein, 37 aa, Artificial Sequence
  Synthetic peptide
QVLGDLNGDKQVNSTDYTALKRHLLNITRLSGTALAN

SEQ ID NO: 14 - protein, 40 aa, Artificial Sequence
  Synthetic peptide
VIPGDVNGDGRVNSSDLTLMKRYLLKSIDFDFPTPEGKIA

SEQ ID NO: 15 - protein, 35 aa, Artificial Sequence
  Synthetic peptide
VVTGDVNGDGNVNSTDLTILKRYLLKSVTNINREA

SEQ ID NO: 16 - protein, 36 aa, Artificial Sequence
  Synthetic peptide
VTYGDVNGDGRVNSSDLALLKRYLLGLVENINKEAA

SEQ ID NO: 17 - protein, 39 aa, Artificial Sequence
  Synthetic peptide
VLYGDVNDDGKVNSTDLTLLKRYVLKAVSTLPSSKAEKA

SEQ ID NO: 18 - protein, 38 aa, Artificial Sequence
  Synthetic peptide
IKHGDLNFDNAVNSTDLLMLKRYILKSLELGTSEHEEK

SEQ ID NO: 19 - protein, 35 aa, Artificial Sequence
  Synthetic peptide
VVYGDLNNDSKVNAVDIMMLKRYILGIIDNINLTA

SEQ ID NO: 20 - protein, 35 aa, Artificial Sequence
  Synthetic peptide
KLYGDVNDDGKVNSTDAVALKRYVLRSGISINTDN

SEQ ID NO: 21 - protein, 37 aa, Artificial Sequence
  Synthetic peptide
PLKGDVNGDGHVSSDYSLFKRYLLRVIDRFPVGDQSV

SEQ ID NO: 22 - protein, 38 aa, Artificial Sequence
  Synthetic peptide
ADVDGDQQITALDFSLIKQYLLGTINKFPAQTASKIKP

SEQ ID NO: 23 - protein, 23 aa, Artificial Sequence
  Synthetic peptide
ADMNNDGNIDAIDFAQLKVKLLN

SEQ ID NO: 24 - protein, 25 aa, Artificial Sequence
  Synthetic peptide
ADMNGDGAIDAIDYALLKKALLANQ

SEQ ID NO: 25 - protein, 30 aa, Artificial Sequence
  Synthetic peptide
ADLNGDGKVDSTDLMILHRYLLGIISSFPR

SEQ ID NO: 26 - protein, 29 aa, Artificial Sequence
  Synthetic peptide
ADVNRDGKVDSTDLMMLHRYLLRIISKLG

SEQ ID NO: 27 - protein, 25 aa, Artificial Sequence
  Synthetic peptide
ADLNEDGKVNSTDLLALKKLVLREL

SEQ ID NO: 28 - protein, 30 aa, Artificial Sequence
  Synthetic peptide
ADVNRDGAINSSDMTILKRYLIKSIPHLPY

SEQ ID NO: 29 - protein, 30 aa, Artificial Sequence
  Synthetic peptide
ADVNVSGTVNSTDLAIMKRYVLRSISELPY

SEQ ID NO: 30 - protein, 30 aa, Artificial Sequence
  Synthetic peptide
ADVNRDGRVNSSDVTILSRYLIRVIEKLPI

SEQ ID NO: 31 - protein, 30 aa, Artificial Sequence
  Synthetic peptide
ADLNRDNKVDSTDLTILKRYLLYAISEIPI

SEQ ID NO: 32 - protein, 30 aa, Artificial Sequence
  Synthetic peptide
ADIYFDGVVNSSDYNIMKRYLLKAIEDIPY

SEQ ID NO: 33 - protein, 30 aa, Artificial Sequence
  Synthetic peptide
ADLNEDGRVNSDLGILKRYILKEIDTLPKN

SEQ ID NO: 34 - protein, 28 aa, Artificial Sequence
  Synthetic peptide
ADVNRDGRIDSTDLTMLKRYLIRAVPSL

SEQ ID NO: 35 - protein, 59 aa, Artificial Sequence
  Synthetic peptide
GDVNGDGTINSTDLTMLKRSVLRAITLTDDAKARADVDKNGSINSTDVLLLSRYLLRVI

SEQ ID NO: 36 - protein, 65 aa, Artificial Sequence
  Synthetic peptide
GDVNDDGKVNSTDAVALKRYVLRSGISINTDNADLNEDGRVNSTDLGILKRYILKEIDTLPYKNG

SEQ ID NO: 37 - protein, 64 aa, Artificial Sequence; identical to SEQ ID NO: 1
  Synthetic peptide

SEQ ID NO: 38 - protein, 64 aa, Artificial Sequence; identical to SEQ ID NO: 1
  Synthetic peptide

SEQ ID NO: 39 - protein, 63 aa, Artificial Sequence
  Synthetic peptide
GDLNGDKQVNSTDYTALKRHLLNITRLSGTALANADVNRDGKVDSTDLMMLHRYLLRIISKLG

SEQ ID NO: 40 - protein, 75 aa, Artificial Sequence
  Synthetic peptide
GDLNGDKQVNSTDYTALKRHLLNITRLSGTALANADLNGDGKVDSTDLMILHRYLLGIISSFPRSNPQPSSNPQP

SEQ ID NO: 41 - protein, 63 aa, Artificial Sequence
  Synthetic peptide
GDCNGDGKVNSTDAVALKRYILRSGISINTDNADVNADGRVNSTDLAILKRYILKEIDVLPHK

SEQ ID NO: 42 - protein, 63 aa, Artificial Sequence
  Synthetic peptide
GDVNGDGHVNSSDYSLFKRYLLRVIDRFPVGDQSVADVNRDGRIDSTDLTMLKRYLIRAIPSL

SEQ ID NO: 43 - protein, 73 aa, Artificial Sequence
  Synthetic peptide
GDLNNDSKVNAVDIMMLKRYILGIIDNINLTAADIYFDGVVNSSDYNIMKRYLLKAIEDIPYVPENQAPKAIF

SEQ ID NO: 44 - protein, 63 aa, Artificial Sequence; identical to SEQ ID NO: 39
  Synthetic peptide

SEQ ID NO: 45 - protein, 75 aa, Artificial Sequence; identical to SEQ ID NO: 40
  Synthetic peptide

SEQ ID NO: 46 - protein, 67 aa, Artificial Sequence
  Synthetic peptide
GDLNGDGKVNSTDLTIMKRYILKNFDKLAVPEEAADLNGDGRINSTDLSILHRYLRRIITSFPVEQQ

SEQ ID NO: 47 - protein, 63 aa, Artificial Sequence; identical to SEQ ID NO: 41
  Synthetic peptide

SEQ ID NO: 48 - protein, 63 aa, Artificial Sequence
  Synthetic peptide
GDVNGDGRVNSSDVALLKRYLLGLVENINKEAADVNVSGTVNSTDLAIMKRYVLRSISELPYK

SEQ ID NO: 49 - protein, 66 aa, Artificial Sequence
  Synthetic peptide
GDVNDDGKVNSTDLTLLKRYVLKAVSTLPSSKAEKNADVNRDGRVNSSDVTILSRYLIRVIEKLPI

SEQ ID NO: 50 - protein, 49 aa, Artificial Sequence
  Synthetic peptide
GDVNGDGHVNSSDYSLFKRYLLRVIDRFPVGDPQDGCGRHDRVVDSGSK

SEQ ID NO: 51 - protein, 63 aa, Artificial Sequence; identical to SEQ ID NO: 42
  Synthetic peptide

SEQ ID NO: 52 - protein, 63 aa, Artificial Sequence
  Synthetic peptide
GDCNDDGKVNSTDVAVMKRYLKKENVNINLDNADVNADGKVNSTDFSILKRYVMKNIEELPYR

SEQ ID NO: 53 - protein, 75 aa, Artificial Sequence
  Synthetic peptide
GDVNGDGTINSTDLTMLKRSVLRAITLTDDAKARADVDKNGSINSTDVLLLSRYLLRVIDKFPVAENPSSSFKYE

SEQ ID NO: 54 - protein, 68 aa, Artificial Sequence
  Synthetic peptide
GDLNGDGKVNSSDLAILKRYMLRAISDFPIPEGRKLADLNRDGNVNSTDYSILKRYILKAIDNIPVDD

SEQ ID NO: 55 - protein, 67 aa, Artificial Sequence
  Synthetic peptide
GDLNGDGKVNSTDLTIMKRYILKNFDKLAVPEEAADLNGDGRINSTDLSILHRYLLRIITSFPVEQQ

SEQ ID NO: 56 - protein, 63 aa, Artificial Sequence
  Synthetic peptide
GDLNGDGNINSTDFTMLKRAILGNPAPGTNLAAGDLNRDGNTNSTDLMILRRYLLKLIGSLPI

SEQ ID NO: 57 - protein, 62 aa, Artificial Sequence
  Synthetic peptide
GDINLDGKINSTDLSALKRHILRITTLSGKQLENADVNNDGSVNSTDASILKKYIAKAIPSL

SEQ ID NO: 58 - protein, 72 aa, Artificial Sequence
  Synthetic peptide
GDVNGDFAVNSNDLTLIKRYVLKNIDEFPSPHGLKAADVDGNEKITSSDAALVKRYVLRAITSFPVEENQNE

SEQ ID NO: 59 - protein, 67 aa, Artificial Sequence
  Synthetic peptide
GDLNGDNRINSTDLTLMKRYILKSIEDLPVEDDLWAADINGDGKINSTDYTYLKKYLLQAIPELPKK

SEQ ID NO: 60 - protein, 66 aa, Artificial Sequence
  Synthetic peptide
GDLNQDGQVSSTDLVAMKRYLLKNFELSGVGLEAADLNSDGKVNSTDLVALKRFLLKEIDELPLKR

SEQ ID NO: 61 - protein, 71 aa, Artificial Sequence
  Synthetic peptide
GDINLDGKINSSDVTLLKRYIVKSIDVFPTADPERSLIASDVNGDGRVNSTDYSYLKRYVLKIIPTIPGNS

SEQ ID NO: 62 - protein, 66 aa, Artificial Sequence; identical to SEQ ID NO: 49
  Synthetic peptide

SEQ ID NO: 63 - protein, 77 aa, Artificial Sequence
  Synthetic peptide
GDVNGDGKINSTDCTMLKRYILRGIEEFPSPSGIIAADVNADLKINSTDLVLMKKYLLRSIDKFPAEDSQTPDEDNP

SEQ ID NO: 64 - protein, 64 aa, Artificial Sequence
  Synthetic peptide
GDVNADGVINSSDIMVLKRFLLRTITLTEEMLLNADTNGDGAVNSSDFTLLKRYILRSIDSFPV

SEQ ID NO: 65 - protein, 69 aa, Artificial Sequence
  Synthetic peptide
GDINGDNSVNSTDLTILKRYLLGSTVPTAPNWRLAADLNLDGNINSTDFTILKRYILGRIEAPPWVNQT

SEQ ID NO: 66 - protein, 69 aa, Artificial Sequence
  Synthetic peptide
GDVDGNGTVNSTDVNYMKRYLLRQIEEFPYEKALMAGDVDGNGNINSTDLSYLKKYILKLISAFPAETN

SEQ ID NO: 67 - protein, 72 aa, Artificial Sequence
  Synthetic peptide
GDVNGDFAVNSNDLTLIKRYVLKNIDEFPSSHGLKAADVDGDEKITSSDAALVKRYVLRAITSFPVEENQNE

SEQ ID NO: 68 - protein, 60 aa, Artificial Sequence
  Synthetic peptide
GDLNRNGIVNDEDYILLKNYLLRGNKLVIDLNVADVNKDGKVNSTDCLFLKKYILGLITI

SEQ ID NO: 69 - protein, 69 aa, Artificial Sequence
  Synthetic peptide
GDLNFDNAVNSTDLLMLKRYILKSLELGTSEQEEKFKKAADLNRDNKVDSTDLTILKRYLLKAISEIPI

SEQ ID NO: 70 - protein, 62 aa, Artificial Sequence
  Synthetic peptide
GDINDDGNINSTDLQMLKRHLLRSIRLTEKQLLNADTNRDGRVDSTDLALLKRYILRVITTL

SEQ ID NO: 71 - protein, 62 aa, Artificial Sequence
  Synthetic peptide
GDLNGDGNINSTDLQILKKHLLRITLLTGKELSNADVTKDGKVDSTDLTLLKRYILRFVTNF

SEQ ID NO: 72 - protein, 62 aa, Artificial Sequence
  Synthetic peptide
GDLNDDGKVNSTDFQILKKHLLRITLLTGKNLSNADLNKDGKVDSSDLSLMKRYLLQIIPTF

SEQ ID NO: 73 - protein, 65 aa, Artificial Sequence
  Synthetic peptide
GDLNNDGKVNSTDFQLLKMHVLRQELPAGTDLSNADVNRDGKVDSSDCTLLKRYILRVISDFPQN

SEQ ID NO: 74 - protein, 68 aa, Artificial Sequence
  Synthetic peptide
GDLNGDGKVNSTDLQLMKMHVLRQRQLTGTSLLNADVNRDGKVDSTDVALLKRYILRQISSFDDYARS

SEQ ID NO: 75 - protein, 65 aa, Artificial Sequence
  Synthetic peptide
GDINNDKTVNSTDVTYLKRFLLKQINSLPNQKAADVNLDGNINSTDLVILKRYVLRGISKLPYAP

SEQ ID NO: 76 - protein, 67 aa, Artificial Sequence
  Synthetic peptide
GDYNGDGAVNSTDLLACKRYLLYALKPEQINVIAGDLDGNGKINSTDYAYLKRYLLKQIDKFPVQLK

SEQ ID NO: 77 - protein, 66 aa, Artificial Sequence
  Synthetic peptide
GDLNGDGNVNSTDSTLMSRYLLGIITTLPAGEKAADLNGDGKVNSTDYNILKRYLLKYIDKFPVES

SEQ ID NO: 78 - protein, 69 aa, Artificial Sequence
  Synthetic peptide
GDLNGDNNVNSTDLTLLKRYLTRVINDFPHPDGSVNADVNGDGKINSTDYSAMIRYILRIIDKFPAEKS

SEQ ID NO: 79 - protein, 64 aa, Artificial Sequence
  Synthetic peptide
GDLNGDGLVNSSDYSLLKRYILKQIDLTEEKLKAADLNRNGSVDSVDYSILKRFLLKTITQLPV

SEQ ID NO: 80 - protein, 75 aa, Artificial Sequence
  Synthetic peptide
GDLNNDGRTNSTDYSLMKRYLLGSISFTNEQLKAADVNLDGKVNSSDYTVLRRFLLGSIDLLPYNGTATYQAEDA

SEQ ID NO: 81 - protein, 75 aa, Artificial Sequence
  Synthetic peptide
GDLNGDGNINSSDLQALKRHLLGISPLTGEALLRADVNRSGKVDSTDYSVLKRYILRIITEFPGQGDVQTPNPSV

SEQ ID NO: 82 - protein, 75 aa, Artificial Sequence; identical to SEQ ID NO: 53
  Synthetic peptide

SEQ ID NO: 83 - protein, 67 aa, Artificial Sequence
  Synthetic peptide
GDLNGDGRVNSTDYTLLKRYLLGAIQTFPYERGIKAADLNLDGRINSTDYTVLKRYLLNAIPSLPVK

SEQ ID NO: 84 - protein, 67 aa, Artificial Sequence; identical to SEQ ID NO: 59
  Synthetic peptide

SEQ ID NO: 85 - protein, 63 aa, Artificial Sequence
  Synthetic peptide
GDLNGDGKINSTDISLMKRYLLKQIVDLPVEDDIKAADINKDGKVNSTDMSILKRVILRNYPL

SEQ ID NO: 86 - protein, 76 aa, Artificial Sequence
  Synthetic peptide
GDVNLDGSVDSIDLALLYNTTYYAVPLPNRLQYIAADVNYDSSCTMLDFYMLEDYLLGRISSFPAGQTYTVYYGDL

SEQ ID NO: 87 - protein, 75 aa, Artificial Sequence; identical to SEQ ID NO: 40
  Synthetic peptide

SEQ ID NO: 88 - protein, 64 aa, Artificial Sequence
  Synthetic peptide
GDLNYDGKVNSTDYLVLKRYLLGTIDKESDPNFLKAADLNRDGRVNSTDMSLMKRYLLGIITSF

SEQ ID NO: 89 - protein, 68 aa, Artificial Sequence
  Synthetic peptide
GDVNGDGKVNSTDCSIVKRYLLKNIEDFPYEYGKEAGDVNGDGKVNSTDYSLLKRFVLRNIDKFPVEQ

SEQ ID NO: 90 - protein, 65 aa, Artificial Sequence
  Synthetic peptide
GDINSDGNVNSTDLGILKRIIVKNPPASANMDAADVNADGKVNSTDYTVLKRYLLRSIDKLPHTT

SEQ ID NO: 91 - protein, 77 aa, Artificial Sequence; identical to SEQ ID NO: 63
  Synthetic peptide

SEQ ID NO: 92 - protein, 58 aa, Artificial Sequence
  Synthetic peptide
GDVNLDGQVNSTDFSLLKRYILKVVDINSINVTNADMNNDGNINSTDISILKRILLRN

SEQ ID NO: 93 - protein, 73 aa, Artificial Sequence
  Synthetic peptide
GDINRDGKINSTDLGMLNRHILKLVILDDNLKLAAADIDGNGNINSTDYSWLKKYILKVISEFPGGDTRIVTP

SEQ ID NO: 94 - protein, 66 aa, Artificial Sequence; identical to SEQ ID NO: 49
  Synthetic peptide

SEQ ID NO: 95 - protein, 64 aa, Artificial Sequence
  Synthetic peptide
GDLNGDGRVNSTDLAVMKRYLLKQVQISDIRPADLNGDGKANSTDYQLLKRYILKTIDIFPVEK

SEQ ID NO: 96 - protein, 75 aa, Artificial Sequence
  Synthetic peptide
GDVNADGKIDSTDLTLLKRYLLRSATLTEEKILNADTDGNGTVNSTDLNYLKKYILRVISVFPAEGNKPPTPTPT

SEQ ID NO: 97 - protein, 77 aa, Artificial Sequence
  Synthetic peptide
GDVNADGVVNISDYVLMKRYILRIIADFPADDDMWVGDVNGDNVINDIDCNYLKRYLLHMIREFPKNSYNSAPTFTP

SEQ ID NO: 98 - protein, 65 aa, Artificial Sequence
  Synthetic peptide
GDLNGDGRVNSSDLALMKRYVVKQIEKLNVPVKAADLNGDDKVNSTDYSVLKRYLLRSIEVIPIK

SEQ ID NO: 99 - protein, 63 aa, Artificial Sequence; identical to SEQ ID NO: 52
  Synthetic peptide

SEQ ID NO: 100 - protein, 63 aa, Artificial Sequence; identical to SEQ ID NO: 41
  Synthetic peptide

SEQ ID NO: 101 - protein, 62 aa, Artificial Sequence
  Synthetic peptide
GDVNGDGNVNSTDVVWLRRFLLKLVEDFPVPSGKQAADMNDDGNINSTDMIALKRKVLKIPI

SEQ ID NO: 102 - protein, 67 aa, Artificial Sequence; identical to SEQ ID NO: 55
  Synthetic peptide

SEQ ID NO: 103 - protein, 64 aa, Artificial Sequence
  Synthetic peptide
GDLNGDQKVTSTDYTMLKRYLMKSIDRFNTSEQAADLNRDGKINSTDLTILKRYLLYSIPSLPI

SEQ ID NO: 104 - protein, 66 aa, Artificial Sequence
  Synthetic peptide
GDLNGDGVVNSTDSVILKRHIIKFSEITDPVKLKAADLNGDGNINSSDVSLMKRYLLRIIDKFPVE

SEQ ID NO: 105 - protein, 73 aa, Artificial Sequence; identical to SEQ ID NO: 43
  Synthetic peptide

SEQ ID NO: 106 - protein, 64 aa, Artificial Sequence
  Synthetic peptide
GDIVLDGNINSLDMMKLKKYLIRETQFNYDELLRADVNSDGEVNSTDYAYLKRYILRIIDAFPQ

SEQ ID NO: 107 - protein, 64 aa, Artificial Sequence; identical to SEQ ID NO: 1
  Synthetic peptide

SEQ ID NO: 108 - protein, 61 aa, Artificial Sequence
  Synthetic peptide
GDVNGDGRVNSSDLTLMKRYLLKSISDFPTPEGKIAADLNEDGKVNSTDLLALKKLVLREL

SEQ ID NO: 109 - protein, 65 aa, Artificial Sequence
  Synthetic peptide
GDLNGDGRVNSTDLLLMKKRIIREIDKFNVPDENADLNLDGKINSSDYTILKRYVLKSIEKLPVK

SEQ ID NO: 110 - protein, 63 aa, Artificial Sequence; identical to SEQ ID NO: 48
  Synthetic peptide

SEQ ID NO: 111 - protein, 71 aa, Artificial Sequence
  Synthetic peptide
GDVNFDGRINSTDYSRLKRYVIKSLEFTDPEEHQKFIAAADVDGNGRINSTDLYVLNRYILKLIEKFPAEQ

SEQ ID NO: 112 - protein, 71 aa, Artificial Sequence; identical to SEQ ID NO: 61
  Synthetic peptide

SEQ ID NO: 113 - protein, 62 aa, Artificial Sequence; identical to SEQ ID NO: 57
  Synthetic peptide

SEQ ID NO: 114 - protein, 63 aa, Artificial Sequence; identical to SEQ ID NO: 56
  Synthetic peptide

SEQ ID NO: 115 - protein, 67 aa, Artificial Sequence
  Synthetic peptide
GDLNNDGNINSTDYMILKKYILKVLERMNVPEKAADLNGDGSINSTDLTILKRFIMKAITKFPVTQK

SEQ ID NO: 116 - protein, 69 aa, Artificial Sequence
  Synthetic peptide
GDVNKDGRINSTDIMYLKGYLLRNSAFNLDEYGLMAADVDGNGSVSSLDLTYLKRYILRRISDFPANKK

SEQ ID NO: 117 - protein, 66 aa, Artificial Sequence; identical to SEQ ID NO: 60
  Synthetic peptide

SEQ ID NO: 118 - protein, 64 aa, Artificial Sequence
  Synthetic peptide
GDTNSDGKINSTDVTALKRHLLRVTQLTGDNLANADVNGDGNVNSTDLLLLKRYILGEIENFPI

SEQ ID NO: 119 - protein, 80 aa, Artificial Sequence
  Synthetic peptide
GDLNVDGSINSVDITYMKRYLLRSISVLPYQENERIRIPAADTNGDGAINSSDMVLLKRYVLRSISEFPVKYDIYGNIIN

SEQ ID NO: 120 - protein, 68 aa, Artificial Sequence; identical to SEQ ID NO: 54
  Synthetic peptide

SEQ ID NO: 121 - protein, 62 aa, Artificial Sequence
  Synthetic peptide
GDVNGDGNVNSTDLTMLKRYLLKSVTNINREAADVNRDGAINSSDMTILKRYLIKSIPHLPY

SEQ ID NO: 122 - protein, 63 aa, Artificial Sequence; identical to SEQ ID NO: 42
  Synthetic peptide

SEQ ID NO: 123 - protein, 455 aa, Artificial Sequence
  Synthetic peptide
MGSGDVNDDGKVNSTDAVALKRYVLRSGISINTDNADLNEDGRVNSTDLGILKRYILKEIDTLPYKNEFKNADSYAKKPHISALNAPQLDQRYKNEFTIGAAVEPYQLQNEKDVQMLKRHFNSIVAENVMKPISIQPEEGKFNFEQADRIVKFAKANGMDIRFHTLVWHSQVPQWFFLDKEGKPMVNETDPVKREQNKQLLLKRLETHIKTIVERYKDDIKYWDVVNEVVGDDGKLRNSPWYQIAGIDYIKVAFQAARKYGGDNIKLYMNDYNTEVEPKRTALYNLVKQLKEEGVPIDGIGHQSHIQIGWPSEAEIEKTINMFAALGLDNQITELDVSMYGWPPRAYPTYDAIPKQKFLDQAARYDRLFKLYEKLSDKISNVTFWGIADNHTWLDSRADVYYDANGNVVVDPNAPYAKVEKGKGKDAPFVFGPDYKVKPAYWAIIDHLEHHHHHH

SEQ ID NO: 124 - protein, 438 aa, Artificial Sequence
  Synthetic peptide
MGSKRYVLRSGISINTDNADLNEDGRVNSTDLGILKRYILKEIDTLPYKNEFKNADSYAKKPHISALNAPQLDQRYKNEFTIGAAVEPYQLQNEKDVQMLKRHFNSIVAENVMKPISIQPEEGKFNFEQADRIVKFAKANGMDIRFHTLVWHSQVPQWFFLDKEGKPMVNETDPVKREQNKQLLLKRLETHIKTIVERYKDDIKYWDVVNEVVGDDGKLRNSPWYQIAGIDYIKVAFQAARKYGGDNIKLYMNDYNTEVEPKRTALYNLVKQLKEEGVPIDGIGHQSHIQIGWPSEAEIEKTINMFAALGLDNQITELDVSMYGWPPRAYPTYDAIPKQKFLDQAARYDRLFKLYEKLSDKISNVTFWGIADNHTWLDSRADVYYDANGNVVVDPNAPYAKVEKGKGKDAPFVFGPDYKVKPAYWAIIDHLEHHHHHH

SEQ ID NO: 125 - protein, 315 aa, Artificial Sequence
  Synthetic peptide
MGSGDVNDDGKVNSTDAVALKRYVLRSGISINTDNADLNEDGRVNSTDLGILKRYILKEIDTLPYKNEFMSKGEELFTGVVPILVELDGDVNGHKFSVSGEGEGDATYGKLTLKFICTTGKLPVPWPTLVTTLTYGVQCFSRYPDHMKRHDFFKSAMPEGYVQERTISFKDDGNYKTRAEVKFEGDTLVNRIELKGIDFKEDGNILGHKLEYNYNSHNVYITADKQKNGIKANFKIRHNIEDGSVQLADHYQQNTPIGDGPVLLPDNHYLSTQSALSKDPNEKRDHMVLLEFVTAAGITHGMDELYKLEHHHHHH

SEQ ID NO: 126 - protein, 298 aa, Artificial Sequence
  Synthetic peptide
MGSKRYVLRSGISINTDNADLNEDGRVNSTDLGILKRYILKEIDTLPYKNEFMSKGEELFTGVVPILVELDGDVNGHKFSVSGEGEGDATYGKLTLKFICTTGKLPVPWPTLVTTLTYGVQCFSRYPDHMKRHDFFKSAMPEGYVQERTISFKDDGNYKTRAEVKFEGDTLVNRIELKGIDFKEDGNILGHKLEYNYNSHNVYITADKQKNGIKANFKIRHNIEDGSVQLADHYQQNTPIGDGPVLLPDNHYLSTQSALSKDPNEKRDHMVLLEFVTAAGITHGMDELYKLEHHHHHH

SEQ ID NO: 127 - protein, 312 aa, Artificial Sequence
  Synthetic peptide
MSHHHHHHMSKGEELFTGVVPILVELDGDVNGHKFSVSGEGEGDATYGKLTLKFICTTGKLPVPWPTLVTTLTYGVQCFSRYPDHMKRHDFFKSAMPEGYVQERTISFKDDGNYKTRAEVKFEGDTLVNRIELKGIDFKEDGNILGHKLEYNYNSHNVYITADKQKNGIKANFKIRHNIEDGSVQLADHYQQNTPIGDGPVLLPDNHYLSTQSALSKDPNEKRDHMVLLEFVTAAGITHGMDELYKVVPDVNDDGKVNSTDAVALKRYVLRSGISINTDNADLNEDGRVNSTDLGILKRYILKEIDTLPYKN

SEQ ID NO: 128 - protein, 296 aa, Artificial Sequence
  Synthetic peptide
MSHHHHHHMSKGEELFTGVVPILVELDGDVNGHKFSVSGEGEGDATYGKLTLKFICTTGKLPVPWPTLVTTLTYGVQCFSRYPDHMKRHDFFKSAMPEGYVQERTISFKDDGNYKTRAEVKFEGDTLVNRIELKGIDFKEDGNILGHKLEYNYNSHNVYITADKQKNGIKANFKIRHNIEDGSVQLADHYQQNTPIGDGPVLLPDNHYLSTQSALSKDPNEKRDHMVLLEFVTAAGITHGMDELYKVVPKRYVLRSGISINTDNADLNEDGRVNSTDLGILKRYILKEIDTLPYKN

SEQ ID NO: 129 - protein, 243 aa, Artificial Sequence
  Synthetic peptide
MGSKRYVLRSGISINTDNADLNEDGRVNSTDLGILKRYILKEIDTLPYKNEFMADTLLILGDSLSAGYRMSASAAWPALLNDKWQSKTSVVNASISGDTSQQGLARLPALLKQHQPRWVLVELGGNDGLRGFQPQQTEQTLRQILQDVKAANAEPLLMQIRLPANYGRRYNEAFSAIYPKLAKEFDVPLLPFLMEEVYLKPQWMQDDGIHPNRDAQPFIADWMAKQLQPLVNHDSLEHHHHHH

SEQ ID NO: 130 - protein, 508 aa, Artificial Sequence
  Synthetic peptide
MGSKRYVLRSGISINTDNADLNEDGRVNSTDLGILKRYILKEIDTLPYKNEFMSKITFPKDFIWGSATAAYQIEGAYNEDGKGESIWDRFSHTPGNIADGHTGDVACDHYHRYEEDIKIMKEIGIKSYRFSISWPRIFPEGTGKLNQKGLDFYKRLTNLLLENGIMPAITLYHWDLPQKLQDKGGWKNRDTTDYFTEYSEVIFKNLGDIVPIWFTHNEPGVVSLLGHFLGIHAPGIKDLRTSLEVSHNLLLSHGKAVKLFREMNIDAQIGIALNLSYHYPASEKAEDIEAAELSFSLAGRWYLDPVLKGRYPENALKLYKKKGIELSFPEDDLKLISQPIDFIAFNNYSSEFIKYDPSSESGFSPANSILEKFEKTDMGWIIYPEGLYDLLMLLDRDYGKPNIVISENGAAFKDEIGSNGKIEDTKRIQYLKDYLTQAHRAIQDGVNLKAYYLWSLLDNFEWAYGYNKRFGIVHVNFDTLERKIKDSGYWYKEVIKNNGFLEHHHHHH

SEQ ID NO: 131 - protein, 198 aa, Artificial Sequence
  Synthetic peptide
MGSKRYVLRSGISINTDNADLNEDGRVNSTDLGILKRYILKEIDTLPYKNEFLVPRGSMVDNKFNKEQQNAFYEILHLPNLNEEQRNAFIQSLKDDPSQSANLLAEAKKLNDAQAPKVDNKFNKEQQNAFYEILHLPNLNEEQRNAFIQSLKDDPSQSANLLAEAKKLNDAQAPKAAAGGDYKDDDDKLALEHHHHHH

SEQ ID NO: 132 - protein, 33 aa, Artificial Sequence
  Synthetic peptide
CCCCATGGGATCCGGCGACGTCAATGATGACGG

SEQ ID NO: 133 - protein, 30 aa, Artificial Sequence
  Synthetic peptide
GGGGAATTCGTTCTTGTACGGCAATGTATC

SEQ ID NO: 134 - protein, 46 aa, Artificial Sequence
  Synthetic peptide
GATACATTGCCGTACAAGAACGAATTCATGAGTAAAGGAGAAGAAC

SEQ ID NO: 135 - protein, 48 aa, Artificial Sequence
  Synthetic peptide
CTCAGTGGTGGTGGTGGTGGTGCTCGAGTTTGTAGAGCTCATCCATGC

SEQ ID NO: 136 - protein, 48 aa, Artificial Sequence
  Synthetic peptide
ACCATGAGCCACCATCACCATCACCATATGAGTAAAGGAGAAGAACTT

SEQ ID NO: 137 - protein, 48 aa, Artificial Sequence
  Synthetic peptide
GTCATCATTGACGTCGCCAGGTACCACTTTGTAGAGCTCATCCATGCC

SEQ ID NO: 138 - protein, 34 aa, Artificial Sequence
  Synthetic peptide
CCATCAGAATTCATGGCGGACACGTTATTGATTC

SEQ ID NO: 139 - protein, 32 aa, Artificial Sequence
  Synthetic peptide
CAGATACTCGAGTGAGTCATGATTTACTAAAG

SEQ ID NO: 140 - protein, 32 aa, Artificial Sequence
  Synthitic peptide
GTATCCGAATTCATGTCAAAGATAACTTTCCC

SEQ ID NO: 141 - protein, 33 aa, Artificial Sequence
  Synthetic peptide
GCATAACTCGAGAAAACCGTTGTTTTTGATTAC

SEQ ID NO: 142 - protein, 35 aa, Artificial Sequence
  Synthetic peptide
GTACAAGAACGAATTCATGTCAAAGATAACTTTCC

SEQ ID NO: 143 - protein, 37 aa, Artificial Sequence
  Synthetic peptide
GGTGGTGGTGCTCGAGAAAACCGTTGTTTTTGATTAC

SEQ ID NO: 144 - protein, 33 aa, Artificial Sequence
  Synthetic peptide
CACGGTGAATTCCTGGTGCCACGCGGTTCCATG

SEQ ID NO: 145 - protein, 33 aa, Artificial Sequence
  Synthetic peptide
CCAATGCTCGAGTGCAAGCTTGTCATCGTCGTC

SEQ ID NO: 146 - protein, 34 aa, Artificial Sequence
  Synthetic peptide
TATACCATGGGATCCAAGAGATATGTTTTGAGAT

SEQ ID NO: 147 - protein, 40 aa, Artificial Sequence
  Synthetic peptide
TCTTGAATTCGTTCTTGTACGGCAATGTATCTATTTCTTT

SEQ ID NO: 148 - protein, 46 aa, Artificial Sequence
  Synthetic peptide
GYVLRSGISINTDNADLNEDGRVNSTDLGILKRYILKEIDTLPYKN

SEQ ID NO: 149 - protein, 44 aa, Artificial Sequence
  Synthetic peptide
GLRSGISINTDNADLNEDGRVNSTDLGILKRYILKEIDTLPYKN

SEQ ID NO: 150 - protein, 42 aa, Artificial Sequence
  Synthetic peptide
GSGISINTDNADLNEDGRVNSTDLGILKRYILKEIDTLPYKN

SEQ ID NO: 151 - protein, 40 aa, Artificial Sequence
  Synthetic peptide
GISINTDNADLNEDGRVNSTDLGILKRYILKEIDTLPYKN

SEQ ID NO: 152 - protein, 38 aa, Artificial Sequence
  Synthetic peptide
GINTDNADLNEDGRVNSTDLGILKRYILKEIDTLPYKN

(19) United States
(12) Patent Application Publication
(10) Pub. No.: US 2011/0151538 A1
(43) Pub. Date: Jun. 23, 2011

(54) AFFINITY PURIFICATION BY COHESIN-DOCKERIN INTERACTION

(75) Inventors: Edward A. Bayer, Ramot Hashavim (IL); Alon Karpol, Rehovot (IL); Raphael Lamed, Yavne (IL)

(73) Assignees: RAMOT AT TEL-AVIV UNIVERSITY LTD., Tel-Aviv (IL); YEDA RESEARCH AND DEVELOPMENT CO. LTD. AT THE WEIZMANN INSTITUTE OF SCIENCE, Rehovot (IL)

(21) Appl. No.: 13/056,913

(22) PCT Filed: Aug. 9, 2009
(86) PCT No.: PCT/IL2009/000773

§ 371 Date: Jan. 31, 2011

Related U.S. Application Data
(60) Provisional application No. 61/086,813, filed on Aug. 7, 2008.
(60) Provisional application No. 61/093,371, filed on Sep. 1, 2008.

(51) Int. Cl. C12N 9/96; C07K 1/22; C07K 16/00; C07K 2/00; C07K 1/04; C07K 14/33

(52) U.S. Cl. 435/188; 530/344; 530/391.1; 530/300; 530/345; 530/324

(57) ABSTRACT

The present invention is directed to truncated dockerin polypeptides, recombinant polypeptides and affinity systems comprising the truncated dockerin polypeptide, methods of generating same, and methods of use thereof to purify, isolate, and detect molecules of interest.

### FIELD OF THE INVENTION

The present invention is directed to truncated dockerin polypeptides, recombinant polypeptides and affinity systems comprising the truncated dockerin polypeptide, methods of generating same, and methods of use thereof to purify, isolate, and detect molecules of interest.

### BACKGROUND OF THE INVENTION

### Affinity Chromatography

Affinity chromatography is a preferred separation technique for isolating biologically active compounds. The binding constant of the immobilized ligand to the target biomolecule, being purified in affinity chromatography systems is determined based on the desired selectivity of the column, good column retention, the capacity of the column, and elution conditions. For example, a very high affinity between the ligand and the biomolecule may require harsh elution conditions (such as low/high pH or very high salt concentrations), which may lead to unfolding or denaturation in the case of a recombinant protein. On the other hand, overly weak affinities may be insufficient for efficient retention of the target protein on the column.

One approach that has been utilized is the use of affinity tags. Affinity tags can be fused to any recombinant protein of interest, allowing rapid, facile purification using the affinity properties of the tag, rather than those of the target protein. Some tags are relatively small in size, such as the His tag, which can be fused either at the N- or C-terminus of a recombinant protein for purification using immobilized metal ion affinity chromatography (IMAC). Small affinity tags show minimal interaction with the targeted protein; hence they usually do not disrupt or impair protein activity. Nevertheless, His tags fail to provide a highly specific interaction; the column capacity is consequently low, and impurities frequently contaminate the sample. Immuno-affinity chromatography employing immobilized protein A or protein G is another preferred system for antibody purification. Other affinity chromatography systems employ affinity tags such as maltose-binding protein (MBP) that binds to amylose resins; glutathione-S-transferase (GST) that binds to glutathione-immobilized matrices; and a FLAG™ fusion tag (Asp-Tyr-Lys-Asp-Asp-Asp-Asp-Lys) which binds to an anti-flag antibody, through which the target protein can be eluted under mild conditions. In some cases, these affinity tags are relatively large, sometimes even larger than the target protein of interest.

### Cohesins and Dockerins

Cellulose, the most abundant biopolymer on earth, holds great potential as an energy source for living organisms. Indeed many microorganisms have evolved to grow on cellulose. While many fungi and aerobic bacteria attack the recalcitrant cellulose substrate by secreting different synergistically acting cellulases, some anaerobic bacteria produce a multi-component enzyme complex, the cellulosome, for efficient degradation of cellulose. The most studied cellulosome-producing microbe is Clostridium thermocellum, a thermophilic, anaerobic, gram-positive bacterium that lives solely on cellulose and its degradation products (Bayer et al., 1998). Cellulosomes are defined as multienzyme complexes having high activity against crystalline cellulose and related plant cell wall polysaccharides such as xylan, mannan, and pectin.

As described in Jindou et al., 2004, cellulosomes have been identified and characterized in cellulolytic clostridia such as Clostridium thermocellum (Lamed et al., 1983), C. cellulolyticum (Pages et al., 1996), C. cellulovorans (Doi et al., 1994), C. papyrosolvens (Pohlschroder 1994) C. josui (Kakiuchi et al., 1998), Acetivibrio cellulolyticus (Ding et al., 1999), Bacteroides cellulosolvens (Ding et al., 2000), R. flavefaciens (Kirby et al., 1997), Ruminococcus albus (Ohara et al., 2000), and Clostridium cellobioparum (Lamed et al., 1987). A common feature of the clostridial cellulosomes is that they consist of a large number of catalytic components arranged around noncatalytic scaffolding proteins. The scaffolding proteins have been identified as CipA (or scaffoldin) in C. thermocellum (Gerngross et al., 1993), CipC in C. cellulolyticum (Pages et al., 1999), CbpA in C. cellulovorans (Shoseyov et al., 1992), and CipA in C. josui (Kakiuchi et al., 1998). These proteins fundamentally consist of repetitive noncatalytic domains of about 140 residues, termed cohesin domains, and a carbohydrate-binding module (CBM). For example, C. josui CipA N-terminus is composed of 3 CBMs followed by a hydrophilic domain and six cohesin domains; and C. thermocellum CipA contains a CBM between the second and third of nine repeated cohesin domains and a type II dockerin domain at its C terminus. The amino acid sequences of all the cohesin domains from these bacteria are in many cases highly homologous to each other, especially within the same species. Each cohesin domain is a subunit-binding domain that interacts with a docking domain, called dockerin, of each catalytic component. The dockerin domain contains two segments, also known as conserved duplicated regions (CDRs), each of which contains a Ca²⁺-binding loop and an alpha helix (namely, the calcium binding motif). An additional alpha helix intervenes between the two segments. The alpha helix in each duplicated sequence contains a conserved KR or KK dipeptide. The species-specific attachment of the dockerin module to the cohesin module is mediated via a high affinity Ca²⁺-dependent interaction.

The C. thermocellum cellulosome contains a CBM and a series of nine cohesin modules that anchor the cellulosomal enzymes to the multienzyme complex (Bayer et al., 2004). The various cellulosomal enzymes contain inter alia a conserved dockerin module that binds to the cohesin counterpart. Biochemical and structural studies on the cohesins-dockerin interaction from C. thermocellum scaffoldin have shown that the dockerins can bind to each of the cohesins on the scaffoldin (Yaron et al., 1995) with a strong affinity constant (Mechaly et al., 2000). Binding is typically reversible by addition of divalent ion chelators such as EDTA.

CBMs, which bind to cellulose or chitin, have been utilized in affinity purification. Craig et al., 2006 have described an affinity chromatography system based upon the calcium dependence of the cohesin-dockerin interaction for purification of antibodies. In this system, a CBM-Coh from C. thermocellum was coupled covalently to an activated Sepharose matrix, following which an antibody-binding protein fused to a dockerin module was introduced to the column, and the protein, was eluted with EDTA. Due to the strong cohesin-dockerin interaction, however, the efficiency of the elution step and suitability for repeated use in such studies are unclear.

Fierobe 1999 discloses that the dockerin domain of C. cellulolyticum CipC is sufficient for interaction with cohesin, that removal of the second conserved duplicated region abolishes affinity with cohesin1, and that deletion of the linker sequence immediately preceding the dockerin domain disrupts folding of the domain and sharply reduces affinity for cohesin. Thus, this reference teaches against use of a dockerin domain on the extreme N- or C-terminus of any protein.

Karpol et al., 2008 discloses a series of deletions in the N- and/or C-terminal portions of a C. thermocellum dockerin module fused to a xylanase. The mutated dockerins having deletions in the calcium-coordinating residues exhibited efficient binding to cohesin, which was abolished by EDTA.

US Patent Application No. 2005/0106700 discloses use of C-terminal and N-terminal dockerin fusions in purification of target proteins on affinity columns. International Patent Application No. WO 2009/028532 discloses purification systems and methods using a dockerin polypeptide characterized in that the amino acid at the 14-position in the second sub-domain of a dockerin originating from Clostridium josui is substituted by another amino acid.

None of the prior publications teach or suggest use of the truncated dockerin domains of the present invention in affinity columns or related technologies, their attachment to the N- or C-terminus of a target protein, or the advantageous reversible binding attained thereby.

There remains a need for a cost-effective affinity tag for use in affinity purification systems. Additionally, there remains a need for a purification process capable of obtaining high yields and high purity of protein concentrate without reducing the specific activity of the isolated proteins.

### SUMMARY OF THE INVENTION

The present invention is directed to affinity purification systems using truncated dockerin polypeptides, the truncated dockerin comprising a single calcium binding motif. The present invention further provides recombinant polypeptides comprising the mutated dockerin domain and affinity columns comprising the truncated dockerin polypeptides, methods of generating same, and methods of use thereof to purify, isolate, and detect molecules of interest. It should be appreciated that the molecule of interest may be any type of molecule which it is desirable to purify. According to certain embodiments the molecule of interest is covalently bound to the truncated dockerin domain chemically or in case the molecule of interest is a peptide it may be fused to the truncated dockerin domain to form a recombinant polypeptide.

It is now disclosed for the first time that the mutated dockerin domain is advantageous for use in a cohesin-dockerin purification system due to the ability of the CBM-cohesin polypeptide to bind tenaciously to a cellulose matrix. Furthermore, the system affords facile, cost-effective and efficient regeneration of the column for repeated use.

According to the principles of the present invention a truncated dockerin domain, comprising only one of the two Ca²⁺ binding motifs, exhibited high binding capacities to a cohesin domain that was reduced compared to that of the wild type dockerins. The wild-type dockerin was shown to bind cohesin with an extremely tight association, and thus was found to be unsuitable for affinity chromatography systems. Unexpectedly, truncation of the dockerin domain conferred reversible binding to the cohesin-dockerin system and enabled recovery of more that 90% of an exemplary covalently bound target protein. Protein recovery from columns utilizing the truncated dockerin domain was highly efficient and characterized by high levels of purity in a single step, directly from crude cell extracts. Moreover, the truncated dockerin tag had no significant effect on the activity of the purified enzyme compared to the activity of the wild-type enzyme.

Reference is made to FIG. 3A, which depicts the structure of the intact wild-type C. thermocellum Cel48S dockerin domain (SEQ ID NO: 1) used to design the truncated dockerin domains of the present invention. As shown, dockerin domains begin with a conserved glycine residue, which is designated residue 1 in the numbering used herein. The dockerin domain contains two segments, also known as conserved duplicated regions (CDRs), each of which contains a Ca²⁺-binding loop and an alpha helix (namely, the calcium binding motif). An additional alpha helix intervenes between the two segments referred to in the figure as duplicated sequence 1 and duplicated sequence 2. Each duplicated sequence forms a single calcium binding motif. The alpha helix in each duplicated sequence of the Cel48S dockerin domain contains a conserved KR or KK dipeptide. In other dockerin domains the conserved dipeptide may consist of KR, KK, KY, KM, KN, SR, RR, or KG. According to certain embodiments, the truncated dockerin domain of the present invention lacks 14-16 amino acids N-terminal to said conserved dipeptide.

It will be apparent to those skilled in the art that based on the present disclosure, dockerin analogues of the truncated dockerin polypeptides of the present invention can be made in dockerin domains other than that of Cel48S. FIG. 11, for example, provides a sequence alignment of the two dockerin segments of multiple species (SEQ ID NO: 10-SEQ ID NO: 34; starting from the −3 position, according the numbering utilized herein). FIG. 12 (SEQ ID NO:35-SEQ ID NO: 122) further provides a sequence alignment of the dockerin domains from C. thermocellum. According to certain embodiments, the C. thermocellum Type-I dockerin domain has the amino acid sequence as set forth in any one of SEQ ID NO:35-SEQ ID NO:122, or an analog or derivative thereof. According to certain embodiments, the dockerin domain is from a thermophilic microorganism. According to certain embodiments the thermophilic microorganism is selected from a group consisting of Clostridium thermocellum and Archaeoglobus fulgidus. Each possibility represents a separate embodiment of the present invention.

According to one aspect, the present invention provides an affinity purification system comprising a solid substrate, a bound protein comprising a cohesin domain, and a recombinant polypeptide comprising a molecule of interest and a truncated dockerin polypeptide derived from a dockerin domain, the truncated dockerin comprising only one calcium binding motif.

In some embodiments, said molecule of interest is a molecule other than a protein. In other embodiments the molecule of interest is a peptide or polypeptide. In another embodiment, said molecule of interest is selected from the group consisting of a peptide, an enzyme, a hormone and an antibody. In another embodiment, said molecule of interest is an enzyme other than xylanase. Each possibility represents a separate embodiment of the present invention.

In another embodiment, said molecule of interest is an antibody-binding moiety, and said affinity purification system further comprises the at least the antigen binding portion of an antibody bound to the antibody-binding moiety. According to certain embodiments, an antibody-binding moiety is attached to an affinity column of the present invention via fusion of the antibody-binding moiety to a truncated dockerin polypeptide. The truncated dockerin polypeptide is preferably able to reversibly attach to a cohesin-containing protein bound to the affinity column. Preferably, the antibody-binding moiety is selected from the group consisting of an anti-IgG antibody, protein A, protein G, and protein L. The affinity column can thus be used as a column for purifying a ligand recognized by the bound antibody. In another embodiment said molecule of interest is a ligand, wherein the affinity column can thus be used as a column for binding and/or purifying antibodies that bind specifically to the ligand of choice. Each possibility represents a separate embodiment of the present invention.

In one embodiment, the solid substrate is selected from the group consisting of a bead, a cell, an extracellular matrix, and a container. In another embodiment, the solid substrate is an affinity resin. In another embodiment, the solid substrate is an affinity column. In another embodiment, an affinity column of methods and compositions of the present invention comprises cellulose, and the protein bound to the affinity column further comprises a carbohydrate-binding module (CBM). In another embodiment, the means of attachment of the protein to the affinity column is via interaction between the CBM and the cellulose. In another embodiment, the dockerin domain is a Type-I dockerin domain. In another embodiment, said truncated dockerin polypeptide contains a 14-16 amino acid deletion N-terminal to the lysine residue at position 18 of the wild-type Type I dockerin domain sequence. In another embodiment, said truncated dockerin polypeptide further comprises an N-terminal glycine residue, wherein the glycine residue is attached directly to the truncated dockerin polypeptide. In another embodiment, the cohesin domain on the bound protein is a Type I cohesin domain, capable of interacting with the truncated dockerin domain attached to the molecule of interest. Each possibility represents a separate embodiment of the present invention.

According to a further aspect, the present invention provides a recombinant or synthetic polypeptide comprising a molecule of interest and a truncated dockerin polypeptide derived from a dockerin domain, wherein the truncated dockerin polypeptide comprises only one calcium binding motif. According to certain embodiments, the dockerin domain is a Type-I dockerin domain. According to some embodiments, the Type-I dockerin domain has the amino acid sequence as set forth in any one of SEQ ID NO:1, SEQ ID NO:35-SEQ ID NO:122, or an analog or derivative thereof. According to certain embodiments, the analog comprises at least 70% homology to SEQ ID NO:1. In another embodiment, said truncated dockerin polypeptide contains a 14-16 amino acid deletion N-terminal to the lysine residue at position 18 of the wild-type Type I dockerin domain sequence. Each possibility represents a separate embodiment of the present invention.

In some embodiments, said molecule of interest is a molecule other than a protein. In other embodiments, said molecule of interest is selected from the group consisting of a peptide, an enzyme, a hormone and an antibody. In another embodiment, said molecule of interest is a molecule other than xylanase. Each possibility represents a separate embodiment of the present invention.

In another embodiment, the truncated dockerin polypeptide is linked to the N-terminus of the peptide. In another embodiment, the molecule of interest is a peptide, said truncated dockerin polypeptide is linked to the C-terminus of said peptide. In another embodiment, said truncated dockerin polypeptide is linked to said molecule of interest via a peptide bond. In another embodiment, said truncated dockerin polypeptide is linked to said molecule of interest via a linker peptide. In another embodiment, the linker peptide is a cleavable linker peptide. In another embodiment, the cleavable linker peptide is self-cleavable. Each possibility represents a separate embodiment of the present invention.

In another embodiment, said molecule of interest is an enzyme. In another embodiment, said molecule of interest is an antibody-binding moiety bound to an antibody. Preferably, the antibody-binding moiety is selected from the group consisting of an anti-IgG antibody, protein A, protein G, and protein L. Each possibility represents a separate embodiment of the present invention.

According to a further aspect, the present invention provides a method of attaching a molecule of interest to a solid substrate, the method comprising the step of: a) providing a solid substrate associated with a protein comprising a cohesin domain; b) providing a molecule of interest covalently bound to a truncated dockerin polypeptide, wherein the truncated dockerin polypeptide comprises only one calcium binding motif; c) allowing the truncated dockerin molecule to bind to the cohesin domain; thereby attaching a molecule of interest to a solid substrate. In one embodiment, the molecule of interest is a fusion peptide. In another embodiment, said molecule of interest is a molecule other than a peptide. In another embodiment, said molecule of interest is a molecule other than xylanase. Each possibility represents a separate embodiment of the present invention.

In one embodiment, the step of attaching a molecule of interest to a solid substrate is performed in the presence of Ca²⁺. As provided herein, methods of the present invention enable attachment of proteins of solid substrates that is readily reversible under non-denaturing conditions. In one embodiment, said solid substrate is selected from the group consisting of a bead, a cell, an extracellular matrix, and a container. In another embodiment, said solid substrate comprises cellulose and said protein associated with said solid substrate further comprises a carbohydrate-binding module (CBM). In one embodiment, the cohesin domain is a Type I cohesin domain. In one embodiment, the dockerin domain is a Type I dockerin domain. In another embodiment, said truncated dockerin polypeptide contains a 14-16 amino acid deletion N-terminal to the lysine residue at position 18 of the wild-type Type I dockerin domain sequence. In another embodiment, said truncated dockerin polypeptide further comprises an N-terminal glycine residue, wherein the glycine residue is attached directly to the truncated dockerin polypeptide. Each possibility represents a separate embodiment of the present invention.

According to another aspect, the present invention provides a method of purifying a molecule of interest, the method comprising the steps of: a) providing a solid substrate associated with a protein comprising a cohesin domain; b) providing a molecule of interest covalently bound to a truncated dockerin polypeptide, wherein the truncated dockerin polypeptide comprises only one calcium binding motif; c) allowing the truncated dockerin molecule to bind to the cohesin domain; and d) eluting the molecule of interest of (b); thereby purifying a molecule of interest.

In one embodiment, the dockerin domain is a Type I dockerin domain. In another embodiment, said truncated dockerin polypeptide contains a 14-16 amino acid deletion N-terminal to the lysine residue at position 18 of the wild-type Type I dockerin domain sequence. In another embodiment, said truncated dockerin polypeptide further comprises an N-terminal glycine residue, wherein the glycine residue is attached directly to the truncated dockerin polypeptide. In one embodiment, the cohesin domain is a Type I cohesin domain. In another embodiment, said solid substrate is selected from the group consisting of a bead, a cell, an extracellular matrix, and a container. In another embodiment, said solid substrate comprises cellulose and said protein associated with said solid substrate further comprises a carbohydrate-binding module. Each possibility represents a separate embodiment of the present invention.

In another embodiment, the step of attaching a molecule of interest to a solid substrate is performed in the presence of Ca²⁺. In another embodiment, the step of eluting the molecule of interest is performed with a chelator of a divalent cation. In another embodiment, the chelator is selected from the group consisting of EDTA and EGTA. Each possibility represents a separate embodiment of the present invention.

In certain embodiments, said molecule of interest is a molecule other than a peptide. In other embodiments, said molecule of interest is a molecule other than xylanase. Each possibility represents a separate embodiment of the present invention. In one embodiment, the molecule of interest is a fusion peptide. In another embodiment, the molecule of interest is fused to the truncated dockerin polypeptide via a cleavable linker peptide, the method further comprising the step of cleaving said cleavable linker peptide. In another embodiment, said cleavable linker peptide is self-cleavable. Each possibility represents a separate embodiment of the present invention.

In another embodiment, the present invention provides a method of purifying a molecule of interest, the method comprising the steps of: a) providing a solid substrate associated with a protein comprising a cohesin domain; b) providing a truncated dockerin polypeptide covalently bound to an antibody-binding domain bound to an antibody, the antibody recognizes the molecule of interest, wherein the truncated dockerin polypeptide comprises only one calcium binding motif; c) allowing the truncated dockerin molecule to bind to the cohesin domain; and d) eluting the molecule of interest; thereby purifying a molecule of interest.

In one embodiment, the antibody-binding moiety is selected from the group consisting of an anti-IgG antibody, protein A, a protein G, and a protein L. Each possibility represents a separate embodiment of the present invention.

In another embodiment, the present invention provides a method of purifying a molecule of interest, the method comprising the steps of: (a) contacting a solid substrate with the molecule of interest; and (b) eluting the molecule of interest; wherein the solid substrate comprises (i) a first protein bound thereto, wherein the first protein comprises a cohesin domain; and (ii) a second protein or domain fused to the truncated dockerin polypeptide of the present invention, wherein the second protein or domain recognizes the molecule of interest, thereby purifying a molecule of interest. In another aspect, the present invention provides a method of engineering a molecule of interest to be readily purified using a solid substrate, the method comprising the step of fusing the molecule of interest to a truncated dockerin polypeptide, the truncated dockerin polypeptide is derived from a dockerin domain and comprises only one calcium binding motif, thereby engineering a molecule of interest to be purified using an affinity column.

In one embodiment, the dockerin domain is a Type-I dockerin domain. In another embodiment, the molecule of interest is a peptide. In one embodiment, the truncated dockerin polypeptide of methods of the present invention is linked to the C-terminus of the peptide. In another embodiment, the truncated dockerin polypeptide of methods of the present invention is linked to the N-terminus of said peptide.

According to another aspect, the present invention provides an isolated truncated dockerin polypeptide derived from a dockerin domain, wherein the truncated dockerin polypeptide comprises only one calcium binding motif, an analog, derivative and fragment thereof. According to one embodiment, the isolated truncated polypeptide comprises from about 35 to about 70 amino acids. According to another embodiment, the isolated polypeptide comprises from about 40 to about 55 amino acids. According to certain embodiments, said truncation involves deletion of a 14-16 amino acid fragment. Each possibility represents a separate embodiment of the present invention.

According to certain embodiments, the dockerin domain is a Type-I dockerin protein domain. According to some embodiments, the Type-I dockerin domain has the amino acid sequence as set forth in SEQ ID NO: 1 (GDVNDDGKVNSTDAVALKRYVLRSGISINTDNADLNEDGRVNSTDLGILKRYIL KEIDTLPYKN), or an analog or derivative thereof According to certain embodiments, the analog comprises at least 70% homology to SEQ ID NO: 1. Each possibility represents a separate embodiment of the present invention.

According to certain embodiments, the truncated dockerin polypeptide of the present invention comprises only one calcium binding motif, wherein the retained calcium binding motif is in the first segment of the dockerin domain. According to this embodiment, said truncation comprises the calcium-coordinating residues in the second segment of the dockerin domain. According to certain embodiments, said truncation involves deletion of a 14-16 amino acid fragment. According to certain embodiments, the truncated dockerin polypeptide comprises the amino acid sequence as set forth in SEQ ID NO: 4, an analog or fragment thereof Each possibility represents a separate embodiment of the present invention.

According to other embodiments, the retained calcium binding motif of the isolated polypeptide is in the second segment of the dockerin domain. According to this embodiment, said truncation comprises the calcium-coordinating residues in the first segment of the dockerin domain. According to certain embodiments, said truncation involves deletion of a 14-16 amino acid fragment. According to certain embodiments, said truncated dockerin polypeptide contains a 14-16 amino acid deletion N-terminal to the lysine residue at position 18 of the wild-type Type I dockerin domain sequence. According to certain embodiments, the truncated dockerin polypeptide comprises the amino acid sequence as set forth in any one of SEQ ID NO: 2, SEQ ID NO: 3 and SEQ ID NO: 6, an analog or fragment thereof According to one embodiment, the truncated dockerin polypeptide consists of the amino acid sequence as set forth in SEQ ID NO: 2. According to another embodiment, the truncated dockerin polypeptide consists of the amino acid sequence as set forth in SEQ ID NO: 3. Each possibility represents a separate embodiment of the present invention.

In another embodiment, the present invention provides an isolated polynucleotide sequence encoding the truncated dockerin polypeptide of the present invention. In another embodiment, the present invention provides an expression vector comprising the isolated polynucleotide sequence encoding the truncated dockerin polypeptide of the present invention. In another embodiment, the present invention provides a host cell comprising the expression vector. Each possibility represents a separate embodiment of the present invention.

Further embodiments and the full scope of applicability of the present invention will become apparent from the detailed description given hereinafter. However, it should be understood that the detailed description and specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE FIGURES

FIG. 1. Schematic representation of the cohesin-dockerin-based affinity purification approach. CBM-Coh is first bound to the beaded cellulose resin. The dockerin-bearing target protein is then applied and eluted subsequently using increasing concentrations of EDTA.

FIG. 2. Affinity purification of wild-type Doc-Xyn on a CBM-Coh affinity column. (A) Performance of the affinity column. (B) SDS-PAGE analysis of the chromatogram, Cell lysate and CBM-Coh before and after the elution.

FIG. 3. Progressive truncation of dockerin derivatives borne at the N-terminus of the target protein. (A) Sequences of the wild type dockerin domain (SEQ ID NO:1) and the truncated dockerin derivatives. (B) Comparative binding of the tructated dockerins to cohesin. (C) Changes in binding free energies (AAG) upon truncation, calculated as the ratio between the wild type and the mutant dockerins. (D) Comparison of eluted His-tagged Doc(Δ16)-Xyn, purified using either IMAC or on a CBM-Coh column (Coh-Doc). (E) Calcium-dependent binding properties of the truncated and the wild type Doc-Xyn measured using the ELISA-based assay. (▪) and (▾) indicate wtDoc-Xyn supplemented with 1 mM CaCl2 or 10 mM EDTA, respectively. (▴) and (♦) indicate Doc(Δ16)-Xyn supplemented with 1 mM CaCl2 or 10 mM EDTA, respectively.

FIG. 4. Affinity purification of truncated Doc(Δ16)-Xyn on a CBM-Coh affinity column. (A) Performance of the affinity column through repeated application (Ap) and elution (El) of the target protein. (B) SDS-PAGE analysis of the chromatogram. The protein profiles of the crude cell lysate and the CBM-Coh are also shown.

FIG. 5. (A) Determination of relative binding affinity by competitive ELISA. Microtiter plates were coated with a CBM-Coh and interacted with either wtDoc-GFP (▪) or Doc(Δ16)-GFP (▴) in the presence of competitor wtDoc-Xyn. wtDoc-Xyn was used as a control(▾). Measured OD values reflect the relative amount of wtDoc Xyn bound to the coating cohesin. (B) Repeated purification of Doc(Δ16)-GFP on a CBM-Coh affinity column. (C 1-3) Consecutive elution fractions were analyzed by SDS-PAGE in order to evaluate protein purity. (D) Evaluation of the flow-through fraction after consecutive applications. (E) Boiled column beads after final wash. (F) SDS-PAGE analysis of Doc(Δ16)-GFP fusion protein purified on Ni-NTA. Gel visualization was done using coomassie brilliant blue staining.

FIG. 6. SDS-PAGE analysis of wtDoc-GFP fusion protein purified on CBM-Coh or Ni-NTA column. (A) Demonstration of the elution fraction by applying increasing amount of EDTA, and after boiling the column beads with SDS. The flow-through of the unbound cell lysate is shown as well. (B) Ni-NTA elution fractions.

FIG. 7. SDS-PAGE analysis of GFP with C-terminal ΔDoc or wtDoc affinity tag, purified on CBM-Coh. (A-B) Two consecutive elutions of GFP-Doc(Δ16). (C) GFP-wtDoc elution followed by boiling the column beads in order to release attached proteins.

FIG. 8. Doc(Δ16)-ZZ-domain purification and binding activity. (A) SDS PAGE analysis of the CBM-Coh purified Doc(Δ16)-ZZ domain. (B) SDS PAGE analysis of the purified antibodies using immobilized Doc(Δ16)-ZZ.

FIG. 9. Doc(Δ16)-BglA purification and activity assessment. (A) SDS PAGE analysis of the CBM-Coh purified Doc(Δ16)-BglA. (B) Doc(Δ16)-BglA activity curve. (C) wild-type BglA activity curve

FIG. 10. Doc(Δ16)-TEP1 purification and activity assessment. (A) SDS-PAGE analysis of the CBM-Coh purified Doc(Δ16)-TEP1. (B) ΔDoc-TEP1 activity curve.

FIG. 11. Alignment of the amino acid sequences of dockerin domains of Aga27A, Cel8A and Cel48A of C. josui (Cj); and Xyn11A, Xyn11B, Xyn10C, Cel5A, Cel8A, Cel9A, Cel26A-Cel5E (formerly CelH), Cel9D-Cel44A, Cel48A, and Lic16A (formerly LicB) of C. thermocellum (Ct). Asterisks indicate amino acid residue involved in calcium binding. Residues believed to serve as selectivity determinants are indicated by pound signs (#) Amino acids that have conserved similar chemical properties (I, L, M, V, K, R, S, and T) are presented in white on black or black on gray (Jindou, S. et al., 2004). (A) Sequence alignment of the first dockerin segment. (B) Sequence alignment of the second dockerin segment.

FIG. 12. Sequence alignment of dockerin modules from Clostridium thermocellum.

### DETAILED DESCRIPTION OF THE INVENTION

The present invention is directed to truncated dockerin polypeptides, recombinant polypeptides and affinity columns comprising the truncated dockerin polypeptide, methods of generating same, and methods of using same to purify, isolate, and detect molecules of interest.

As exemplified herein below, a truncated dockerin polypeptide lacking one of the two Ca²⁺ binding motifs retained relatively high binding capacities to a cohesion domain. The truncated dockerin polypeptide functioned as an effective affinity tag, and highly purified target proteins were obtained in a single step directly from crude cell extracts. Furthermore, the truncated dockerin affinity tag had no significant effect on the activity of the purified enzyme compared to the activity of the wild-type enzyme.

As disclosed herein below, the affinity column maintained high levels of capacity upon repeated rounds of loading and elution. Further, the coupling of the CBM-Cohesin to the matrix was not achieved by chemical activation of the protein, but by the advantageously innate property of the CBM to bind tenaciously to an inexpensive cellulose matrix.

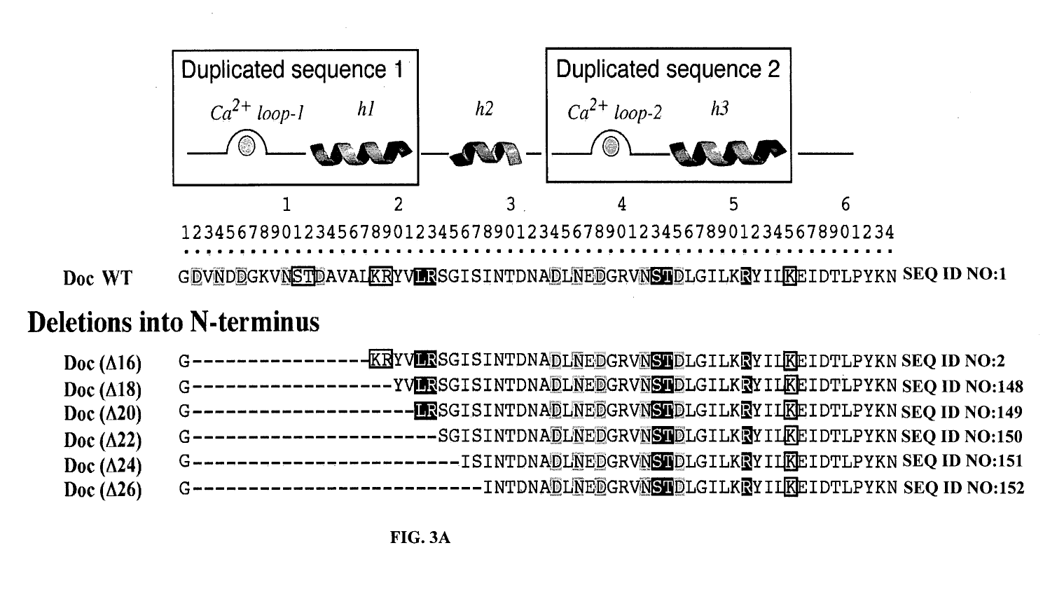

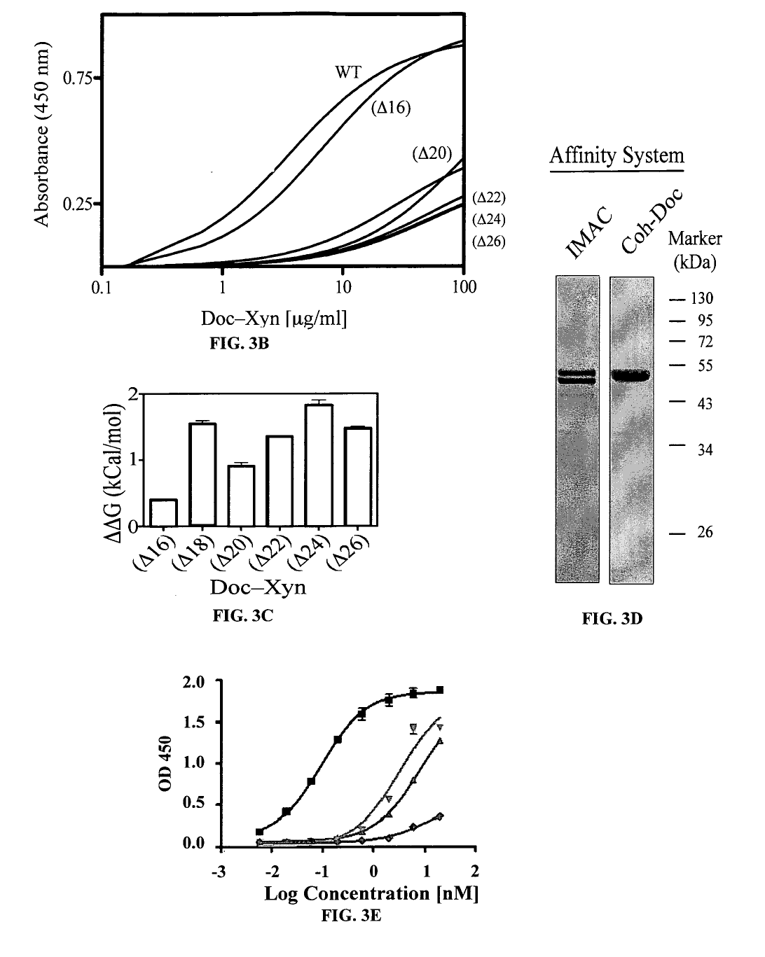

According to one aspect, the present invention provides an affinity purification system, comprising a solid substrate, a bound protein comprising a cohesin domain and a recombinant or synthetic polypeptide comprising a molecule of interest and a truncated dockerin polypeptide derived from a dockerin domain, wherein the truncated dockerin polypeptide comprises only one calcium binding motif. Reference is made to FIG. 3A, which depicts the domain structure of the wild-type dockerin domains used to design the truncated dockerin polypeptides of the present invention. As shown, wild-type dockerin domains begin with a conserved glycine residue, which is designated residue 1 in the numbering used herein. The dockerin domains contain two segments, also known as conserved duplicated regions (CDRs), each of which contains a Ca²⁺-binding loop and an alpha helix. An additional alpha helix intervenes between the two segments referred to in the figure as duplicated sequence 1 and duplicated sequence 2. Each duplicated sequence forms a single calcium binding motif. The alpha helix in each duplicated sequence contains a conserved KR or KK dipeptide. In other dockerin domains the conserved dipeptide may consist of KR, KK, KY, KM, KN, SR, RR or KG. It is exemplified herein below that deletions in the first or second segment of the dockerin domain confer highly advantageous properties in affinity purification systems. The terms “truncated dockerin” and “mutated dockerin domain” are used herein interchangeably and refer to any deletion removing one of the two calcium binding motifs, to provide a dockerin polypeptide comprising only one calcium binding motif.

In one embodiment the truncated dockerin of methods and compositions of the present invention, comprises a 14-16 amino acid deletion N-terminal to the lysine residue at position 18 of the wild-type Type I dockerin domain sequence. In one embodiment, “Deletion N-terminal to the lysine residue at position 18” denotes a deletion wherein 14-16 amino acids between residues 1-17 inclusive are removed. According to preferred embodiments, the 14-16 amino acids deletion comprises the calcium-coordinating residues in the first segment of the dockerin domain. According to further embodiments, the calcium-coordinating residues in the second segment of the dockerin domain are un-mutated. In another embodiment, a truncated dockerin polypeptide of methods and compositions of the present invention begins with residue 17 of the Type I dockerin domain. In another embodiment, the truncated dockerin polypeptide begins with residue 16 of the Type I dockerin domain. In another embodiment, the truncated dockerin polypeptide begins with residue 15 of the Type I dockerin domain. In another embodiment, a truncated dockerin polypeptide of the present invention is defined as a dockerin domain containing a deletion of 14-16 amino acids between residues 2-17 thereof, inclusive. In another embodiment, the deletion is 15-16 amino acids. In another embodiment, the deletion is 16 amino acids. Each possibility represents a separate embodiment of the present invention.

In another embodiment the truncated dockerin of methods and compositions of the present invention, comprises a 14-16 amino acid deletion N-terminal to the lysine residue at position 50 of the wild-type Type I dockerin domain sequence. “Deletion N-terminal to the lysine residue at position 50” is used herein to denote a deletion wherein 14-16 amino acids between residues 34-49 inclusive are removed. According to preferred embodiments, said 14-16 amino acids deletion comprises the calcium-coordinating residues in the second segment of the dockerin domain. According to further embodiments, the calcium-coordinating residues in the first segment of the dockerin domain are un-mutated. In another embodiment, a truncated dockerin polypeptide of methods and compositions of the present invention begins with residue 1 of the Type I dockerin domain. In another embodiment, the truncated dockerin domain begins with residue 2 of the Type I dockerin domain. In another embodiment, a truncated dockerin polypeptide of the present invention is defined as a dockerin domain containing a deletion of 14-16 amino acids between residues 34-49 thereof, inclusive. In another embodiment, the deletion is 15-16 amino acids. In another embodiment, the deletion is 16 amino acids. Each possibility represents a separate embodiment of the present invention.

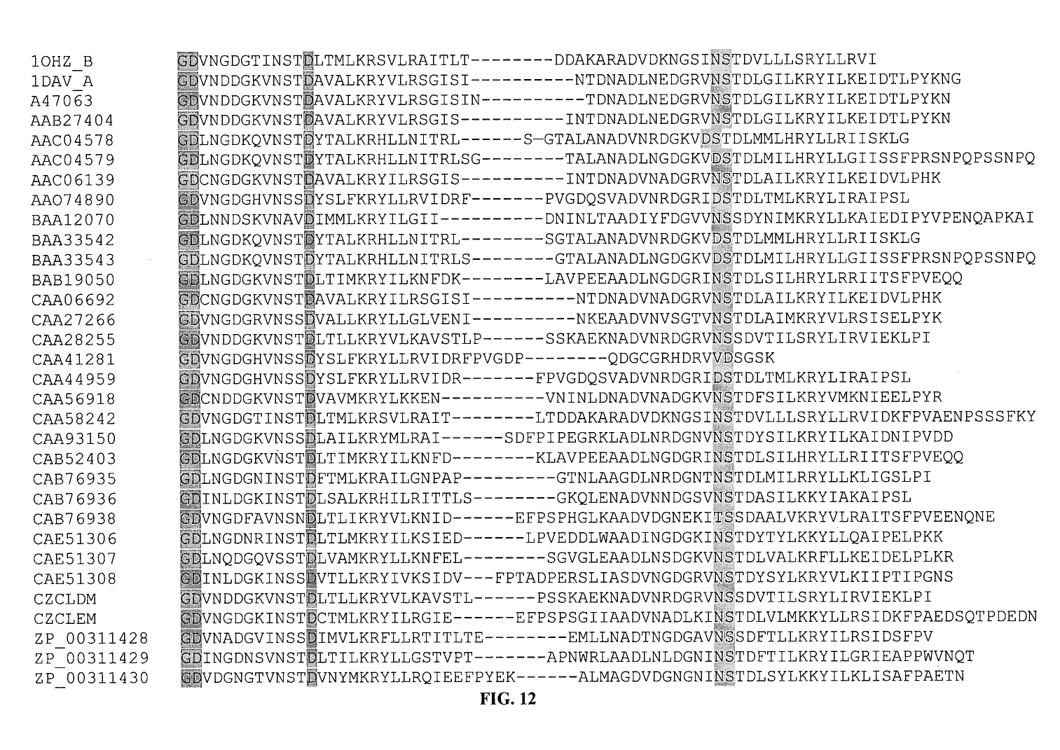

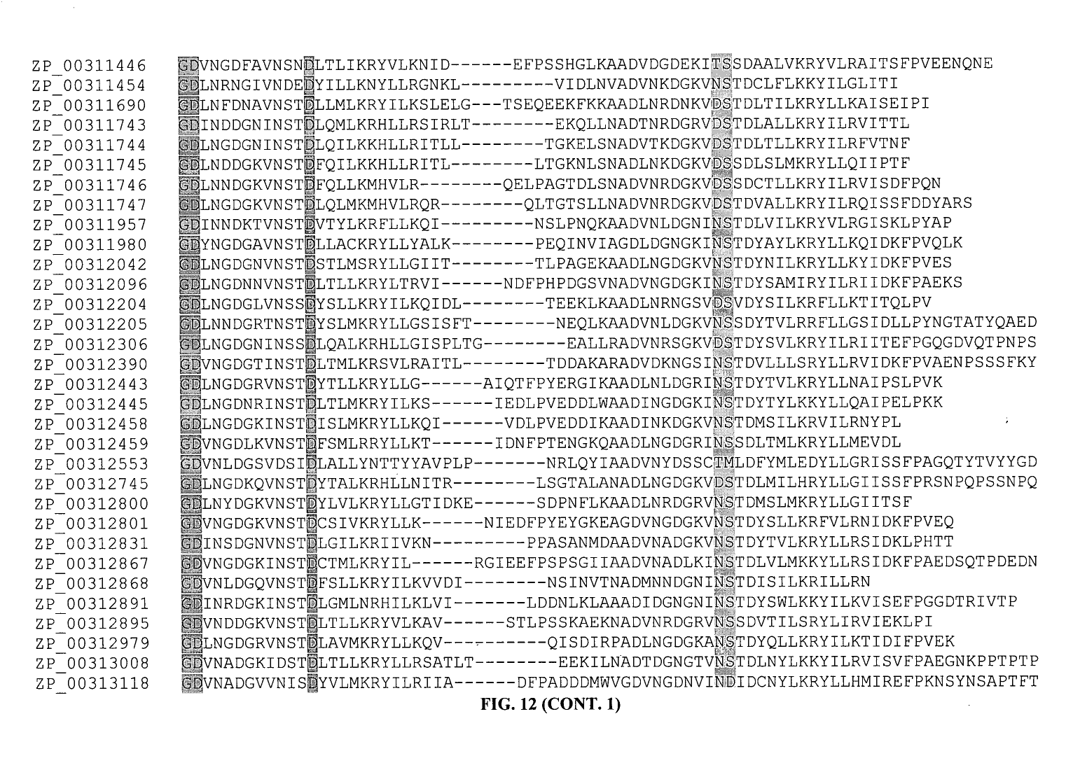

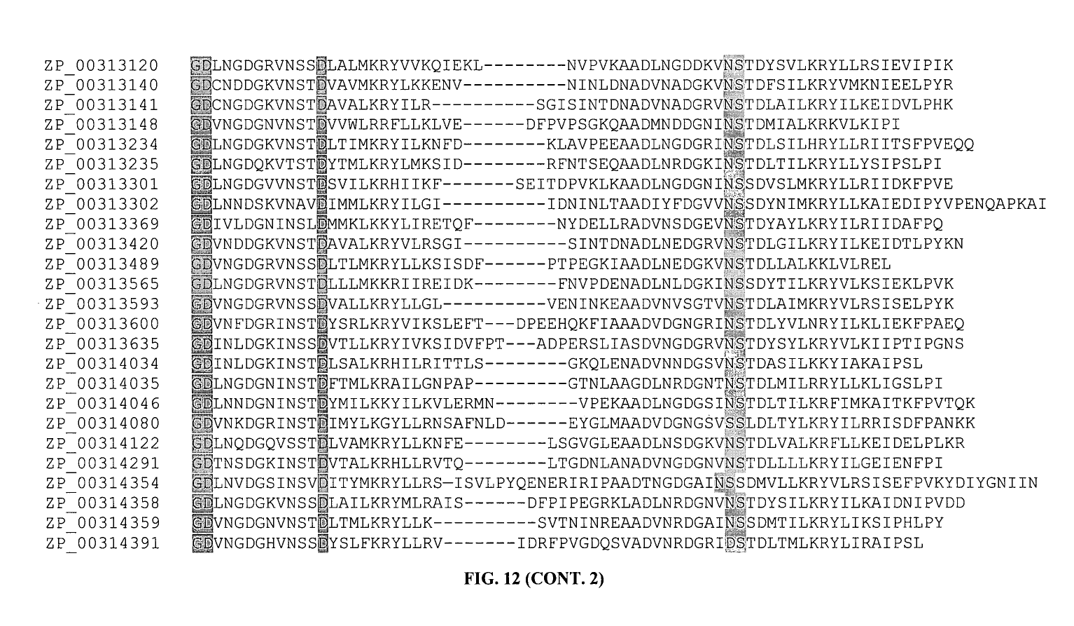

In another embodiment, the wild-type Type I dockerin domain has an amino acid sequence as set forth in SEQ ID NO: 1 (GDVNDDGKVNSTDAVALKRYVLRSGISINTDNADLNEDGRVNSTDLGILKRYIL KEIDTLPYKN), an analog having at least 70% sequence homology to SEQ ID NO: 1 or a fragment thereof. It will be apparent to those skilled in the art that dockerin deletions analogous to the dockerin deletions of the present invention can be made in dockerin proteins other than Cel48S. FIG. 11 (SEQ ID NO:10-SEQ ID NO:34), for example, provides an alignment of the N-terminal approximately 35 amino acids of dockerin domains of multiple species (starting from the −3 position, according the numbering utilized herein). In another embodiment, the wild-type Type I dockerin domain has an amino acid sequence as set forth in any one of SEQ ID NO: 35-SEQ ID NO: 122. FIG. 12 depicts an alignment of additional dockerin domains, wherein the KR or KK dipeptide in the first segment of the dockerin domain is clearly delineated. In certain embodiments, a dipeptide selected from the group consisting of KN, KK, KM, and KG is present instead of KR or KK. The lysine residue in this dipeptide is used instead of the first lysine in KR or KK in designing the truncated dockerin polypeptide. In other embodiments, a dipeptide selected from the group consisting of SR, RR, and NR is present instead of KR or KK. The first residue in this dipeptide is used instead of the first lysine in KR or KK in designing the truncated dockerin polypeptide. Corresponding deletions in other dockerin proteins (namely, 14-16 amino acid deletions N-terminal to but not including the KR, KK, KY, KM, KN, SR, RR, or KG dipeptide in the first or second segments of the dockerin domain) can thus readily be made. In another embodiment, the deletion is 14-16 amino acids, alternatively 15-16 amino acids, further alternatively 16 amino acids between but not including the conserved glycine and the KR, KK, KY, KM, KN, SR, RR, or KG dipeptide in the first segment of the dockerin domain. In another embodiment, the deletion is 15-17 amino acids, alternatively 16-17 amino acids, further alternatively 17 amino acids up to but not including the KR, KK, KY, KM, KN, SR, RR, or KG dipeptide in the first segment of the dockerin domain. In another embodiment, the deletion is 15-17 amino acids, alternatively 16-17 amino acids, further alternatively 17 amino acids up to but not including the KR, KK, KY, KM, KN, SR, RR, or KG dipeptide in the second segment of the dockerin domain. Each possibility represents a separate embodiment of the present invention.

According to a further aspect the present invention provides a recombinant or synthetic polypeptide comprising a molecule of interest covalently bound to a truncated dockerin polypeptide derived from a dockerin protein domain, the truncated dockerin polypeptide comprising only one calcium binding motif.

It will be apparent to those skilled in the art that the truncated dockerin domain of the present invention may be linked to molecule of interest via a direct covalent bond or via a suitable linker. According to some embodiments the molecule of interest is a peptide or polypeptide and the truncated dockerin domain may be linked conveniently to either the N-terminus or the C-terminus, for example via a peptide bond. “Linked to” as utilized herein, refers to connection of two entities via a covalent bond. Linkages between peptide moieties may be via one or more peptide bonds within a polypeptide chain. The term encompasses embodiments wherein a linker peptide is present and wherein the molecule of interest is directly linked via a single peptide bond to a truncated dockerin domain of the present invention. It will be understood by those of skill in the art that such linkage can be performed by engineering a nucleotide molecule to encode a fusion peptide of the present invention or by chemical or other means of directly attaching peptides to one another. According to alternative embodiments the linkage may be performed synthetically to yield non-peptide bonds. Each possibility represents a separate embodiment of the present invention.

In another embodiment, a recombinant peptide of the present invention further comprises an N-terminal glycine residue attached directly to a truncated dockerin domain of the present invention. “Attached directly” as used herein refers to a lack of intervening sequence between the N-terminal glycine residue and the C-terminal dockerin fragment. Truncated dockerin polypeptides of the present invention thus may typically consist of the combination of an N-terminal glycine residue and a C-terminal dockerin domain fragment, wherein the glycine residue is attached directly to the dockerin domain fragment without an intervening, or linker, peptide. In another embodiment, no N-terminal glycine residue is present. Each possibility represents a separate embodiment of the present invention.

“C-terminal Type I dockerin domain fragment” refers to a dockerin fragment that extends until the end of the wild-type dockerin domain. The C-terminus of the dockerin domain is often considered to be the C-terminus of the second segment of said dockerin domain thereof of the wild-type sequence. In another embodiment, in the case of a dockerin domain located on the C-terminus of a protein of interest, the C-terminus of the dockerin domain is considered to be the C-terminus of the molecule of interest. To illustrate this, the C-terminus of the truncated dockerin polypeptide utilized in the Examples, KRYVLRSGISINTDNADLNEDGRVNSTDLGILKRYILKEIDTLPYKN (SEQ ID NO: 5), is the C-terminus of the wild-type sequence SEQ ID NO:1, which is also the C-terminus of the wild-type Cel48S protein (SEQ ID NO: 7; GenBank Accession No. L06942). In another embodiment, a C-terminal dockerin domain fragment of the present invention extends at least until the conserved isoleucine residue occurring shortly after the alpha helix of the second segment of said dockerin domain. This conserved isoleucine residue is the first isoleucine residue after the alpha helix of the second segment and typically is located about two residues thereafter (residue 57 according to the numbering used herein). In the case of the truncated dockerin domain utilized in the Examples, such a peptide would comprise the sequence GKRYVLRSGISINTDNADLNEDGRVNSTDLGILKRYILKEI (SEQ ID NO: 3). In another embodiment, a C-terminal Type I dockerin domain fragment of the present invention extends until the end of a sequence selected from SEQ ID NO:35-SEQ ID NO:122 (FIG. 12). In another embodiment, additional C-terminal residues from the wild-type dockerin domain sequence are included. Each possibility represents a separate embodiment of the present invention.

It is to be understood that the present invention comprises affinity purification systems and methods for purifying a molecule of interest utilizing the novel reversible interaction between a cohesion domain and a truncated dockerin polypeptide of the present invention. Thus, affinity purification system of the present invention comprises, in another embodiment, a recombinant polypeptide comprising a molecule of interest and a cohesion domain, and a bound protein comprising a truncated dockerin polypeptide of the present invention. In another embodiment, the present invention provides a method for purifying a molecule of interest, the method comprises the steps of (a) contacting a solid substrate with a molecule of interest and (b) eluting said molecule of interest, wherein the solid substrate is associated with a protein comprising a truncated dockerin polypeptide of the present invention, and the molecule of interest has been fused to cohesion domain. Each possibility represents a separate embodiment of the present invention.

### Affinity Column Apparatus

The present invention further provides an affinity column apparatus comprising an affinity column, a recombinant polypeptide of the present invention and a bound protein comprising a cohesin domain. Preferably, the cohesin domain on the bound protein is capable of interacting with the recombinant polypeptide comprising a truncated dockerin polypeptide attached to the molecule of interest.

In a preferred embodiment, an affinity column of the present invention comprises cellulose, and the protein bound to the affinity column further comprises a carbohydrate-binding module (CBM). In another embodiment, the means of attachment of the protein to the affinity column is via interaction between the CBM and the cellulose. Each possibility represents a separate embodiment of the present invention.

In another embodiment, an antibody-binding moiety is attached to an affinity column of the present invention via fusion of the antibody-binding moiety to a truncated dockerin polypeptide. The truncated dockerin polypeptide is preferably able to reversibly attach to a cohesin-containing protein bound to the affinity column. In another embodiment, the antibody-binding moiety is selected from the group consisting of an anti-IgG antibody, protein A, a protein G, and a protein L. In another embodiment, the affinity column apparatus further comprises an antibody that binds to the antibody-binding moiety. The affinity column can thus be used as a column for a ligand recognized by the bound antibody. Each possibility represents a separate embodiment of the present invention.

### Solid Substrates Useful in the Present Invention

The solid substrate of methods and compositions of the present invention is, in another embodiment, a bead. In another embodiment, the solid substrate is a cell. In another embodiment, the solid substrate is an extracellular matrix. In another embodiment, the solid substrate is a fibrous matrix. In another embodiment, the solid substrate is a container. In another embodiment, the container is selected from the group consisting of a beaker, a flask, a cylinder, a test tube, a centrifugation tube, Petri dish, a culture dish and a multi-well plate. In another embodiment, the solid substrate is attached to or associated with an affinity column. Each possibility represents a separate embodiment of the present invention.

In another embodiment, an antibody-binding moiety is attached to a solid substrate of the present invention via fusion of the antibody-binding moiety to a truncated dockerin polypeptide. The truncated dockerin polypeptide is able to reversibly attach to a cohesin-containing protein bound to the solid substrate. In another embodiment, the antibody-binding moiety is selected from the group consisting of an anti-IgG antibody, protein A, a protein G, and a protein L. In another embodiment, the solid substrate apparatus further comprises an antibody that binds to the antibody-binding moiety. The solid substrate can thus be used to immobilize or isolate a ligand recognized by the bound antibody. Each possibility represents a separate embodiment of the present invention.

In another embodiment, a solid substrate of methods and compositions of the present invention comprises cellulose, and the protein bound to the solid substrate further comprises a carbohydrate-binding module (CBM). In another embodiment, the means of attachment of the protein to the solid substrate is via interaction between the CBM and the cellulose. Each possibility represents a separate embodiment of the present invention.

Molecules of Interest that can be Attached to Truncated Dockerin Polypeptides of the Present Invention

The molecule of interest of the methods and compositions of the present invention is any molecule that can be bound covalently, either directly or indirectly, to the truncated dockerin domain containing a single calcium binding motif as disclosed herein. In various embodiments, the molecule of interest is any type of molecule which it is desirable to purify or for which it is desirable to engineer an association with a solid substrate.

In certain embodiments the molecule of interest is a peptide. In another embodiments, the molecule of interest is a protein. In another embodiment, the peptide is an enzyme. In another embodiment, the molecule is a peptide hormone. In another embodiment, the molecule is a recombinant peptide. In another embodiment, the molecule of interest is any other type of peptide for which it is desirable to purify or to engineer an association with a solid substrate. Each possibility represents a separate embodiment of the present invention.

As provided herein, a variety of proteins can be successfully purified with high-efficiency under gentle conditions following fusion to truncated dockerin domains of the present invention. As exemplified herein below, Xylanase (Xyn); green fluorescent protein (GFP), β-glucosidase, BglA and ZZ-domain have been successfully purified; maltose-binding protein (MBP), TEP and CprA have been utilized with equally successful results. It is to be understood that the fusion peptides of methods and compositions of the present invention are not those fusion peptides that were disclosed to in Karpol et al., 2008.

### Elution Steps

In another embodiment, a method of the present invention further comprises the step of eluting the polypeptide comprising the molecule of interest or antibody-binding moiety from the affinity column. In another embodiment, the step of eluting is performed with a chelator of a divalent cation. As provided herein, truncated dockerin polypeptides of the present invention are particularly suitable to efficient elution with divalent cations. In another embodiment, a chelator of Ca²⁺ is utilized. In another embodiment, the chelator is selected from the group consisting of EDTA and EGTA. It should be understood that methods of the present invention may further comprise one or more washing steps. Each possibility represents a separate embodiment of the present invention.

### Truncated Dockerin Polypeptides of the Present Invention and Wild-Type Dockerin Domains Useful in the Design Thereof

A dockerin domain of methods and compositions of the present invention is, in another embodiment, a mutated version of a Type I dockerin domain from a species selected from the group consisting of Clostridium thermocellum, C. cellulolyticum, and C. cellulovorans. In another embodiment, the dockerin domain is from a species selected from the group consisting of Clostridium thermocellum, C. cellulolyticum, C. cellulovorans, C. papyrosolvens, C. josui, Acetivibrio cellulolyticus, Bacteroides cellulosolvens, R. flavefaciens, Ruminococcus albus, and Clostridium cellobioparum. Each possibility represents a separate embodiment of the present invention.

In another embodiment, the dockerin domain of methods and compositions of the present invention is from an Archaeoglobus fulgidus protein. An exemplary embodiment of an Archaeoglobus fulgidus dockerin domain is the domain sequence set forth in GenBank Accession number NP_{—}071198 (Bayer et al., 1999).

In another embodiment, the dockerin domain of methods and compositions of the present invention is from a thermophilic microbe. In another embodiment, the thermophilic microbe is selected from the group consisting of C. thermocellum and Archaeoglobus fulgidus. Each possibility represents a separate embodiment of the present invention.

In another embodiment, one of the C. thermocellum dockerins as set forth in any one of SEQ ID NO: 35-SEQ ID NO: 122 (listed in FIG. 12 herein) is utilized to design a truncated dockerin domain of the present invention.

In another embodiment, a cellulosomal dockerin domain is utilized to design a truncated dockerin domain of the present invention. In another embodiment, a Type I dockerin domain from a cellulosomal protein is used. A listing of Type I dockerin domains from cellulosomal proteins is provided in Gold and Martin, 2007. The sequences in this table are available from the United States Department of Energy Joint Genome Institute. The sequence of the complete genome is available as GenBank Accession Number CP000568. Each sequence represents a separate embodiment of the present invention.

In another embodiment, the dockerin utilized to design the truncated dockerin polypeptide of the present invention is a dockerin disclosed in Table 1 of Zverlov V V et al., 2005. Each of the sequences in this table is available from the United States Department of Energy Joint Genome Institute. The sequence of the complete genome is available as GenBank Accession Number CP000568. Each sequence represents a separate embodiment of the present invention.

In another embodiment, a non-cellulosomal dockerin is utilized to design a truncated dockerin domain of the present invention. Non-cellulosomal dockerins are well-known and well-characterized in the art, and include, inter alia, glycoside hydrolases of family 2 (CpGH2), family 31 (CpGH31; GenBank Accession No. YP_{—}695747), family 95 (CpGH95), and family 20 (GenBank Accession No. YP_{—}696057), μ-toxin/NagH (GenBank Accession No. YP_{—}694648), lacZ (GenBank Accession No. YP_{—}695917), fibronectin type III domain-containing protein (GenBank Accession No. ABG82552 and YP_{—}696557), calx-beta domain-containing protein (GenBank Accession No. ABG83106) and the dockerin domains set forth in GenBank Accession Numbers, YP_{—}138060, AAV48354, AAG20133, YP_{—}843398, YP_{—}844001, ABK15364, YP_{—}844005, AAM04927, AAM05172, YP_{—}501678, NP_{—}981324, AAP11768, ZP_{—}00238756, ZP_{—}00238791, AAO81591, YP_{—}001089310, YP_{—}210742, YP_{—}269348, and ABM17463. Each sequence represents a separate embodiment of the present invention.

Methods for identification of dockerin domains are well known in the art, and are described, inter alia, in Zverlov et al., 2005. This publication describes identification of C. thermocellum dockerin domains; however, one of skill in the art could readily apply the same methods using dockerin domain sequences of other organisms to search the genome sequences of other organisms. In another embodiment, a truncated dockerin polypeptide of methods and compositions of the present invention is an internally deleted version of the sequence: GDVNDDGKVNSTDAVALKRYVLRSGISINTDNADLNEDGRVNSTDLGILKRYIL KEIDTLPYKN (SEQ ID NO: 1), which is the C-terminus of the wild-type Cel48S protein (SEQ ID NO: 7; GenBank Accession No. L06942). In another embodiment, the truncated dockerin domain is a internally deleted version of a sequence having at least 70% homology to SEQ ID NO: 1. In another embodiment, the truncated dockerin domain is a internally deleted version of a sequence having at least 80% homology to SEQ ID NO: 1. In another embodiment, the truncated dockerin domain is a internally deleted version of a sequence having at least 90% homology to SEQ ID NO: 1. In another embodiment, the truncated dockerin domain is a internally deleted version of a sequence having at least 92% homology to SEQ ID NO: 1. In another embodiment, the truncated dockerin domain is a internally deleted version of a sequence having at least 95% homology to SEQ ID NO: 1. In another embodiment, the truncated dockerin polypeptide is a internally deleted version of a sequence having at least 98% homology to SEQ ID NO: 1. Each possibility represents a separate embodiment of the present invention.

In another embodiment, a truncated dockerin polypeptide of methods and compositions of the present invention has the sequence: GKRYVLRSGISINTDNADLNEDGRVNSTDLGILKRYILKEIDTLPYKN (SEQ ID NO: 2). In another embodiment, the truncated dockerin polypeptide has at least 70% homology to SEQ ID NO: 2. In another embodiment, the truncated dockerin polypeptide has at least 80% homology to SEQ ID NO: 2. In another embodiment, the truncated dockerin polypeptide has at least 90% homology to SEQ ID NO: 2. In another embodiment, the truncated dockerin polypeptide has at least 92% homology to SEQ ID NO: 2. In another embodiment, the truncated dockerin polypeptide has at least 95% homology to SEQ ID NO: 2. In another embodiment, the truncated dockerin polypeptide has at least 98% homology to SEQ ID NO: 2. Each possibility represents a separate embodiment of the present invention.

In another embodiment, a truncated dockerin polypeptide of methods and compositions of the present invention has the sequence: MGSKRYVLRSGISINTDNAD LNEDGRVNST DLGILKRYIL KEIDTLPYKN (SEQ ID NO: 6).

It is understood in the art that a truncated dockerin polypeptide of methods and compositions of the present invention may be a fragment of the aforementioned dockerin sequences. The term “fragment” as used herein refers to a portion of a polypeptide which retains the activity of the native polypeptide, i.e., use an affinity tag. In one embodiment the fragment has between about 30 to about 60 amino acids, alternatively between about 35 to about 55 amino acids, further between about 40 to about 50 amino acids. Each possibility represents a separate embodiment of the present invention.

In another embodiment, the Ca²⁺-binding residues (Mechaly et al, 2000; also termed as “Ca²⁺-coordinating residues”) are preserved (i.e. are unmutated) in the second conserved duplicated region (i.e. segment) of a truncated dockerin polypeptide of methods and compositions of the present invention. In another embodiment, the DNDND motif at positions 1, 3, 5, 9, and 12 of the second conserved duplicated region is preserved. In another embodiment, the residues DNDD motif at positions 1, 3, 5, and 12 of the second conserved duplicated region is preserved. In another embodiment, the Ca²⁺-binding residues are preserved in the first conserved duplicated region (i.e. segment) of a truncated dockerin polypeptide of methods and compositions of the present invention. Each possibility represents a separate embodiment of the present invention.

In another embodiment, the cohesin recognition residues (Mechaly et al., 2000; and Pages et al., 1997) are preserved (i.e. are unmutated) in the second conserved duplicated region of a truncated dockerin polypeptide of the present invention. In another embodiment, positions 10 and 11 are preserved. In another embodiment, the preserved residues at these positions are ST. In another embodiment, the preserved residues at these positions are SS. In another embodiment, the preserved residues at these positions are AL. In another embodiment, the preserved residues at these positions are AI. In another embodiment, the cohesin recognition residues are preserved (i.e. are unmutated) in the first conserved duplicated region of a truncated dockerin polypeptide of the present invention. Each possibility represents a separate embodiment of the present invention.

Dockerin-Cohesin Pairs that may be Used in Combination in the Present Invention

The dockerin-cohesin pair utilized in methods and compositions of the present invention are, in another embodiment, from the same species. Dockerins have been shown to bind to each of the cohesins on the scaffoldin (Yaron et al., 1995; Pagès et al., 1997); thus, any cohesin from a given species is expected to bind any dockerin from that species. Cohesin-dockerin interactions are in some cases species-specific.

In another embodiment, the dockerin and cohesin domains utilized are from two different species. As a non-limiting example, dockerin polypeptides of C. thermocellum Xyn11A bind to cohesin polypeptides from C. josui CipA (Jindou et al., 2004). The residues involved in determining specificity of the dockerin-cohesin have been publicized in scientific references. Methods for predicting and experimentally confirming whether a given dockerin-cohesin pair will bind to one another are well known in the art, and are described, for example, herein and in Pages et al., 1997; Nakar et al., 2004; Barak et al., 2005; Haimovitz et al., 2008; Mechaly et al., 2000; and Mechaly et al., 2001). As described in these references, the 11th and 12th residues of both segments, among other residues, are involved in determining the binding specificity of dockerin-cohesin pairs.

In other embodiments, the suitability of a given dockerin-cohesin pair for affinity chromatography can be tested by performing affinity chromatography using, for example, xylanse fusion, as described herein. In such a system, the amount of interacting dockerin, indicative of the affinity of the dockerin-cohesin pair, can be determined immunochemically using anti-xylanase primary antibody and HRP-labeled secondary antibodies (Barak Y et al., 2005). In another embodiment, xylanase activity is measured directly using an appropriate substrate (e.g. p-nitrophenyl derivatives of xylobiose or cellobiose), as described in Handelsman et al., 2004.

In another embodiment, the affinity of between the truncated dockerin polypeptide and the cohesin domain is 2-fold less than the affinity between the wild-type dockerin domain and the cohesin domain. In another embodiment, the cohesin-dockerin pair of methods and compositions of the present invention has a Kₐ, when the proteins are unmutated, of 10⁹-10¹³M⁻¹. In another embodiment, the Kₐ, of the unmutated proteins is 10⁸-10¹³M⁻¹. In another embodiment, the Kₐ, of the unmutated proteins is 2×10⁹-10¹³ M⁻¹. In another embodiment, the Kₐ, of the unmutated proteins is 5×10⁹-10¹³ M⁻¹. In another embodiment, the Kₐ, of the unmutated proteins is 10¹⁰-10¹³ M⁻¹. In another embodiment, the Kₐ, of the unmutated proteins is at least 10⁸M⁻¹. In another embodiment, the Kₐ, of the unmutated proteins is at least 2×10⁹ M⁻¹. In another embodiment, the Kₐ, of the unmutated proteins is at least 5×10⁹ M⁻¹. In another embodiment, the Kₐ, of the unmutated proteins is at least 10¹⁰ M⁻¹. Each possibility represents a separate embodiment of the present invention.

In another embodiment, the Kₐ of the truncated dockerin domain with the wild-type cohesin, in the presence of EDTA is low enough to act as a reversible affinity tag. In another embodiment, the Kₐ of this combination is under 10⁷M⁻¹. In another embodiment, the Kₐ of this combination is under 3×10⁶ M⁻¹. In another embodiment, the Kₐ of this combination is under 10⁶M⁻¹. In another embodiment, the Kₐ of this combination is under 3×10⁵ M⁻¹. In another embodiment, the Kₐ of this combination is under 10⁵ M⁻¹. In another embodiment, the Kₐ of this combination is under 3×10⁴ M⁻¹. In another embodiment, the Kₐ of this combination is under 10⁴ M⁻¹. In another embodiment, the Kₐ of this combination is under 5×10³ M⁻¹. In another embodiment, the Kₐ of this combination is under 2×10⁵ M⁻³. In another embodiment, the Kₐ of this combination is under 10³ M⁻¹. In another embodiment, the Kₐ of this combination is under 5×10² M⁻¹. In another embodiment, the Kₐ of this combination is under 2×10² M⁻¹. In another embodiment, the Kₐ of this combination is under 10² M⁻¹. In another embodiment, the Kₐ of this combination is under 5×10¹ M⁻¹. In another embodiment, the Kₐ of this combination is under 2×10¹ M⁻¹. In another embodiment, the Kₐ of this combination is under 10¹ M⁻¹. Each possibility represents a separate embodiment of the present invention.

### Suitable Cohesin Domains

The cohesin domain of methods and compositions of the present invention is, in another embodiment, a Type-I cohesin domain. In another embodiment, the cohesin domain is a Type-II cohesin domain. In another embodiment, the cohesin domain is any other type of cohesin domain known in the art. Each possibility represents a separate embodiment of the present invention.

In another embodiment, the cohesin domain is from a species selected from the group consisting of Clostridium thermocellum, C. cellulolyticum, and C. cellulovorans. In another embodiment, the cohesin domain is from a species selected from the group consisting of Clostridium thermocellum, C. papyrosolvens, and Clostridium cellobioparum. In another embodiment, the cohesin domain is from a species selected from the group consisting of Clostridium thermocellum, C. cellulolyticum, C. cellulovorans, C. papyrosolvens, C. josui, Acetivibrio cellulolyticus, Bacteroides cellulosolvens, R. flavefaciens, Ruminococcus albus, and Clostridium cellobioparum. Each possibility represents a separate embodiment of the present invention.

In another embodiment, the cohesin domain is a cohesin domain from a protein selected from CipA (or scaffoldin) of C. thermocellum, CipC of C. cellulolyticum, CbpA of C. cellulovorans, and CipA of C. josui. Each possibility represents a separate embodiment of the present invention.

In another embodiment, the cohesin domain of methods and compositions of the present invention is from an Archaeoglobus fulgidus protein. Exemplary embodiments of Archaeoglobus fulgidus cohesin domains include the sequences set forth in GenBank Accession numbers NP_{—}071198 and NP_{—}071199. Each sequence represents a separate embodiment of the present invention.

In another embodiment, the cohesin domain of methods and compositions of the present invention is from a thermophilic bacterium. In another embodiment, the thermophilic microbe is selected from the group consisting of C. thermocellum, and Archaeoglobus fulgidus. Each possibility represents a separate embodiment of the present invention.

In another embodiment, a cellulosomal cohesin domain is utilized in methods and compositions of the present invention. In another embodiment, a Type I cohesin domain from a cellulosomal protein is used.

In another embodiment, a non-cellulosomal cohesin domain is utilized in methods and compositions of the present invention. Non-cellulosomal cohesins are well-known and well-characterized in the art, and include, inter alia, the X82 domains from NanJ (GenBank Accession No. YP_{—}694986); the glycoside hydrolases of family 3, 31, 84, and 20 (CpGH3, CpGH31, CpGH84C, and CpGH20, respectively); and NagJ (GenBank Accession No. Q0TR53), all from Clostridium perfringens (Adams J J et al., Structural basis of Clostridium perfringens toxin complex formation. Proc Natl Acad Sci USA. 2008) and the dockerin domains set forth in GenBank Accession Numbers ABE51693, ABE51694, YP_{—}001324319, YP_{—}001324323, ZP_{—}02077900, ZP_{—}02077903, NP_{—}691654, ZP_{—}02845754, ZP_{—}02846450, ZP_{—}02848919, ZP_{—}02849219, YP_{—}695309, YP_{—}210742, EAY29878, ZP_{—}01693353, CAD71804, NP_{—}864128, ABF40998, ABJ82058, ABB32088, ABQ26119, and ABG39560. Each sequence represents a separate embodiment of the present invention.

Exemplary embodiments of cohesin domains useful in methods and compositions of the present invention are found in GenBank Accession numbers YP_{—}001039469, L08665, NZ_ABVG01000001-NZ_ABVG01000046, AB004845, AB025362, AB011057, AY221113, AY221112, and AJ278969. Each sequence represents a separate embodiment of the present invention.

Other exemplary embodiments of C. thermocellum cohesin domains are found in the CipA protein comprising the amino acid sequence as set forth in SEQ ID NO: 8 (GenBank Accession number Q06851). The protein defined by this sequence contains 9 cohesin domains, in residues 29-182, 183-322, 560-704, 724-866, 889-1031, 1054-1196, 1219-1361, 1384-1526, and 1548-1690.

### Cleavable Linkers

In another embodiment, a recombinant polypeptide of methods and compositions of the present invention further comprises a cleavable linker peptide between the truncated dockerin polypeptide and the molecule of interest or antibody-binding moiety. In another embodiment, the cleavable linker peptide is self-cleavable. Each possibility represents a separate embodiment of the present invention.

Cleavable linkers are well known in the art, and are described, inter alia, in Wu W Y et al., 2006 and in United States patent application 2005/0106700, which is incorporated herein by reference. In another embodiment, the cleavable linker is a chemical or enzymatic cleavage site between the target protein and the dockerin. Each possibility represents a separate embodiment of the present invention.

The term “peptide” as used herein encompasses native peptides (degradation products, synthetically synthesized peptides, or recombinant peptides), peptidomimetics (typically, synthetically synthesized peptides), and the peptide analogues peptoids and semipeptoids, and may have, for example, modifications rendering the peptides more stable while in a solution. Such modifications include, but are not limited to: N-terminus modifications; C-terminus modifications; peptide bond modifications, including but not limited to CH₂—NH, CH₂—S, CH₂—S═O, O═C—NH, CH₂—O, CH₂—CH₂, S═C—NH, CH═CH, and CF═CH; backbone modifications; and residue modifications. Methods for preparing peptidomimetic compounds are well known in the art and are specified, for example, in Ramsden, C. A., ed. (1992), Quantitative Drug Design, Chapter 17.2, F. Choplin Pergamon Press, which is incorporated by reference as if fully set forth herein.

Peptide bonds (—CO—NH—) within the peptide may be substituted, for example, by N-methylated bonds (—N(CH3)-CO—); ester bonds (—C(R)H—C—O—O—C(R)—N—); ketomethylene bonds (—CO—CH2-); α-aza bonds (—NH—N(R)—CO—), wherein R is any alkyl group, e.g., methyl; carba bonds (—CH2-NH—); hydroxyethylene bonds (—CH(OH)—CH2-); thioamide bonds (—CS—NH—); olefinic double bonds (—CH═CH—); retro amide bonds (—NH—CO—); and peptide derivatives (—N(R)—CH2-CO—), wherein R is the “normal” side chain, naturally presented on the carbon atom. These modifications can occur at any of the bonds along the peptide chain and even at several (2-3) at the same time.

Natural aromatic amino acids, Trp, Tyr, and Phe, may be substituted for synthetic non-natural acids such as, for instance, tetrahydroisoquinoline-3-carboxylic acid (TIC), naphthylelanine (Nol), ring-methylated derivatives of Phe, halogenated derivatives of Phe, and o-methyl-Tyr.

The term “amino acid” or “amino acids” is understood to include the 20 naturally occurring amino acids; those amino acids often modified post-translationally in vivo, including, for example, hydroxyproline, phosphoserine, and phosphothreonine; and other less common amino acids, including but not limited to 2-aminoadipic acid, hydroxylysine, isodesmosine, nor-valine, nor-leucine, and ornithine. Furthermore, the term “amino acid” includes both D- and L-amino acids. Conservative substitution of amino acids as known to those skilled in the art are within the scope of the present invention. Conservative amino acid substitutions includes replacement of one amino acid with another having the same type of functional group or side chain e.g. aliphatic, aromatic, positively charged, negatively charged. These substitutions may enhance oral bioavailability, penetration into the central nervous system, targeting to specific cell populations and the like. One of skill will recognize that individual substitutions, deletions or additions to peptide, polypeptide, or protein sequence which alters, adds or deletes a single amino acid or a small percentage of amino acids in the encoded sequence is a “conservatively modified variant” where the alteration results in the substitution of an amino acid with a chemically similar amino acid. Conservative substitution tables providing functionally similar amino acids are well known in the art. The following six groups each contain amino acids that are conservative substitutions for one another: 1) Alanine (A), Serine (S), Threonine (T); 2) Aspartic acid (D), Glutamic acid (E); 3) Asparagine (N), Glutamine (Q); 4) Arginine (R), Lysine (K); 5) Isoleucine (I), Leucine (L), Methionine (M), Valine (V); and 6) Phenylalanine (F), Tyrosine (Y), Tryptophan (W). (see, e.g., Creighton, Proteins (1984)).

As used herein, the term “mutation” carries its traditional connotation and refers to a change, inherited, naturally occurring, or introduced, in a nucleic acid or polypeptide sequence, and is used in its sense as generally known to those of skill in the art.

As used herein, the terms “isolated” refers to oligonucleotides substantially free of other nucleic acids, proteins, lipids, carbohydrates, or other materials with which they can be associated, such association being either in cellular material or in a synthesis medium. The term can also applies to polypeptides, in which case the polypeptide will be substantially free of nucleic acids, carbohydrates, lipids, and other undesired polypeptides.

The term “analogs” extends to any functional chemical or recombinant equivalent of the peptides of the present invention, characterised, in a most preferred embodiment, by their possession of at least one of the abovementioned activities. The term “analog” is also used herein to extend to any amino acid derivative of the peptides as described hereinabove. Generally, an analog will possess in one embodiment at least 70% sequence identity, in another embodiment at least 80% sequence identity, in another embodiment at least 90% sequence identity, in another embodiment at least 95% sequence identity, and in yet another embodiment at least 98% sequence identity with the native polypeptide. Percentage sequence identity can be determined, for example, by the Fitch et al. version of the algorithm (Fitch et al., Proc. Natl. Acad. Sci. U.S.A. 80: 1382-1386 (1983)) described by Needleman et al., (Needleman et al., J. Mol. Biol. 48: 443-453 (1970)), after aligning the sequences to provide for maximum homology. Other alignment techniques are disclosed herein below. Amino acid sequence analogs and variants of a polypeptide can be prepared by introducing appropriate nucleotide changes into DNA encoding the polypeptide, or by peptide synthesis. Such analogs include, for example, deletions from, and/or insertions into, and/or substitutions of, residues within the amino acid sequence of the polypeptide of interest. Any combination of deletion, insertion, and substitution is made to arrive at the final construct, provided that the final construct possesses the desired characteristics. The amino acid changes also can alter post-translational processes of the polypeptide, such as changing the number or position of glycosylation sites. Methods for generating amino acid sequence variants of polypeptides are described, for example, in U.S. Pat. No. 5,534,615, incorporated herein by reference.

As used herein, the term “recombinant polypeptide” refers to a polypeptide that has been produced in a host cell which has been transformed or transfected with a nucleic acid encoding the polypeptide, or produces the polypeptide as a result of homologous recombination.

A tagged target protein (e.s., the recombinant polypeptide of the present invention) can be engineered by inserting a nucleic acid sequence encoding a target protein into a vector such that it is flanked on one side by a nucleic acid sequence encoding a tag of the present invention (e.g. SEQ ID NOs: 2, 3 or 4). In one embodiment, the vector comprises the tag sequence and is flanked on one or both sides by a multiple cloning region comprising one or more restriction sites. Factors to be considered when engineering a tagged target protein include, but are not limited to assuring that the nucleic acid sequence encoding a target protein is inserted so that it is contiguous with the nucleic acid sequence encoding a tag of the present invention. Additionally, it is important to ensure that the sequences encoding the tag and the protein are inserted in frame, thereby assuring translation of the desired tagged protein. In one embodiment, the nucleic acid sequence encoding the tag further comprises a stop codon.

A tagged target synthetic protein (e.s., the synthetic polypeptide of the present invention), and fragments thereof, can be chemically synthesized in whole or in part using techniques disclosed herein above. See also, Creighton, (1983) Proteins: Structures and Molecular Principles, W. H. Freeman & Co., New York, N.Y., United States of America, incorporated herein in its entirety.

Alternatively, in accordance with methods disclosed herein and known in the art, expression vectors containing a partial or the entire tag/target protein coding sequence and appropriate transcriptional/translational control signals are prepared. These methods include in vitro recombinant DNA techniques, synthetic techniques, and in vivo recombination/genetic recombination. See e.g., the techniques described throughout Sambrook et al., (1989) Molecular Cloning: A Laboratory Manual, Cold Spring Harbor Laboratory, New York, N.Y., United States of America, and Ausubel et al., (1989) Current Protocols in Molecular Biology, Greene Publishing Associates and Wiley Interscience, New York, N.Y., United States of America, both incorporated herein in their entirety.

A variety of host-expression vector systems can be employed to express a tagged target protein coding sequence. These include, but are not limited to microorganisms such as bacteria transformed with recombinant bacteriophage DNA, plasmid DNA, or cosmid DNA expression vectors containing a truncated dockerin polypeptide coding sequence or a recombinant polypeptide comprising a truncated dockerin and a protein of interest coding sequence; yeast transformed with recombinant yeast expression vectors containing a coding sequence of the polypeptide of the present invention; insect cell systems infected with recombinant virus expression vectors (e.g., baculovirus) containing a coding sequence of the polypeptide of the present invnetion; plant cell systems infected with recombinant virus expression vectors (e.g., cauliflower mosaic virus, CaMV; tobacco mosaic virus, TMV) or transformed with recombinant plasmid expression vectors (e.g., Ti plasmid) containing a coding sequence of the polypeptide of the present invnetion; or animal cell systems. The expression elements of these systems vary in their strength and specificities.

Depending on the host/vector system utilized, any of a number of suitable transcription and translation elements, including constitutive and inducible promoters, can be used in the expression vector. For example, when cloning in bacterial systems, inducible promoters such as pL of bacteriophage .lamda., plac, ptrp, ptac (ptrp-lac hybrid promoter), and the like can be used. When cloning in insect cell systems, promoters such as the baculovirus polyhedrin promoter can be used. When cloning in plant cell systems, promoters derived from the genome of plant cells, such as heat shock promoters, the promoter for the small subunit of RUBISCO, the promoter for the chlorophyll a/b binding protein, or from plant viruses (e.g., the 35S RNA promoter of CaMV; the coat protein promoter of TMV) can be used. When cloning in mammalian cell systems, promoters derived from the genome of mammalian cells (e.g., metallothionein promoter) or from mammalian viruses (e.g., the adenovirus late promoter; the vaccinia virus 7.5 K promoter) can be used. When generating cell lines that contain multiple copies of the tyrosine kinase domain DNA, SV40-, BPV- and EBV-based vectors can be used with an appropriate selectable marker.

The protein to be purified using the method described herein may be produced using recombinant techniques. Methods for producing recombinant proteins are described, e.g., in U.S. Pat. Nos. 5,534,615 and 4,816,567, incorporated herein by reference. In preferred embodiments, the protein of interest is produced in a CHO cell (see, e.g. WO 94/11026). When using recombinant techniques, the protein can be produced intracellularly, in the periplasmic space, or directly secreted into the medium. If the protein is produced intracellularly, as a first step, the particulate debris, either host cells or lysed fragments, may be removed, for example, by centrifugation or ultrafiltration.

The eluted protein preparation may be subjected to additional purification steps either prior to, or after, the affinity chromatography step. Exemplary further purification steps include hydroxylapatite chromatography; dialysis; hydrophobic interaction chromatography (HIC); ammonium sulphate precipitation; anion or cation exchange chromatography; ethanol precipitation; reverse phase HPLC; chromatography on silica; chromatofocusing; and gel filtration.

The protein thus recovered may be formulated in a pharmaceutically acceptable carrier and may be used for various diagnostic, therapeutic or other uses known for such molecules.

The following examples are presented in order to more fully illustrate some embodiments of the invention. They should, in no way be construed, however, as limiting the broad scope of the invention.

### EXAMPLES

### Materials and Methods

### Cloning of Constructs

PCR

Amplification of DNA fragments for cloning purposes was performed using the T-Gradient device (Biometra, Germany). PCR reaction mixtures contained: PfuTurbo (Stratagene, La Jolla, Calif.) or Extaq (Takara Bio Inc. Otsu, Shiga, Japan) polymerase (4 units), dNTPs (2.5 mM each dNTP), reaction buffer, 0.5 μM of each primer (forward and reverse), DNA template, double distilled water (DDW) was added to complete the total volume to 50 μl. PCR was programmed as follows: 30 sec initial-denaturing at 95° C.; followed by 28-35 cycles of: 30 sec of denaturing at 95° C., 30 sec annealing 50-61° C. (mostly 58° C.), 60-150 sec (depending on length of the amplified DNA) polymerization 72° C.; after the last cycle 5 min of polymerization at 72° C. (in order to finish polymerization). DNA samples were purified using a PCR purification kit (Real Biotech Corporation, RBC).

Digestion

PCR samples and plasmids were double-digested at 37° C. for 1-2 hours with the appropriate digestion enzymes and buffers (New England Biolabs Inc. Baverly, Maryland). The required digested DNA fragments (PCR or plasmid) were run and isolated from agarose gel (0.8-1.2%) and purified using a DNA extraction kit (HiYield™ Gel/PCR DNA Extraction kit from RBC).

Ligation and Selection.

The digested DNA fragments were ligated into the appropriate linearized plasmid (original Novagen pET28a) using T4 DNA ligase according to manufacturer recommendation (Fermentas). Ligated samples were transformed into competent E. coli XL1-blue or DH5α strains.

Bacterial Strains

E. coli Strain K-12 DH5α. Genotype: F⁻ end A1 hsd R17 (rₖ⁻, mₖ⁻) sup E44 thi-1 λ⁻ rec A1 gyr A96 rel A1 Δ(arg F⁻ lac ZYA) U 169 ψ80d lacZΔM15.

E. coli Strain K-12 XL1-blue. Genotype: rec A1 end A1 gyr A96 thi-1 hsd R17 (rₖ⁻, mₖ⁻) sup E44 rel A1 lac {F′ pro AB lacI^{q}ZΔM15 Tn 10 (tet^{r})}.

E. coli Strain B BL21(λDE3). F⁻ ompT gal dcm lon hsdS_{B}(r_{B}⁻ m_{B}⁻) λ(DE3 [lacI lacUV5-T7 gene 1 ind1 sam7 nin5])

Transformation

All the E. coli strains mentioned (XL-1, DH5α, BL21(λDE3)) were transformed using the heat-shock technique. Ligation product or plasmid was added to 200 μl of competent cells and left to stand for 10 min on ice. Following one minute of heat-shock at 42° C. the cells were transferred to ice for two additional minutes. One ml of Luria-Bertani (LB) medium was added to the cells for one hour recovery in a 37° C. shaker. Next the cells were centrifuged (14000×g, 30 sec), resuspended in 100 μl LB and plated on 50 μg/ml kanamycin plates. Antibiotic-resistant colonies were isolated and further screened for positive clones trough the colony PCR procedure (similar to the previously described PCR using a bacterial colony as the PCR template). Positive clones were amplified using a Plasmid DNA purification kit (iNtRON biotechnology Inc.) and verified by sequencing.

### Ligation Independent Cloning

Restriction Free Cloning

Restriction Free (RF) cloning was done according to van den Ent et al. The primers were ˜50 bp long, the 28-bp 5′ part was homologues to the insertion vector whereas the remaining ˜20 bp were homologous to the fragment being inserted. In the first step regular PCR was done, as described above, in order to amplify the fragment to be inserted. This fragment contained flanking ends of 28-bp homologues to the vector. In the second step an additional PCR reaction was conducted. The reaction mixture included 100 ng of the PCR product from the previous step, vector DNA (pET28a) 10 ng, 10 mM dNTPs, PfuTurbo (4 units), the total volume was completed to 50 μl with DDW. The PCR included the following steps: Initial denaturation 95° C. 30 sec; followed by 35 cycles of: denaturation 95° C. 30 sec; annealing 55-61° C. 1 min; extension 68° C. 2 min for every kilobase pair (12-15 min). The final extension was done at 72° C. for 10 min. At the end of the PCR 10 μl were removed and supplemented with 1 μl of the methylated DNA restricting endonuclease (DpnI), and its reaction buffer (NEBuffer 4) for 2 hr at 37° C. Subsequently, the 10 μl were transformed to E. coli DH5α.

In-Fusion™.

The primers were designed as recommended in the user manual using the Clontech online tool (http://bioinfo.clontech.com/infusion/ convertPcrPrimersInit.do). The reaction was performed according to the user manual, and contained 200 ng PCR insert, 100 ng of linearized vector (previously restricted), and deionized water to a final volume of 10 μl. The mixture was added to the In-Fusion Dry-Down pellet incubated for 15 min at 37° C., following 15 min at 50° C. Next, the mixture was transferred to ice, diluted with 40 μl of TE buffer and transformed to E. coli DH5α.

### Protein Expression and Purification:

Protein Expression

E. coli BL21(λDE3) strain was used for over-expression of the recombinant proteins. The host cells were grown in 250-500 ml LB medium, supplemented with 50 μg/ml kanamycin, at 37° C. until culture reached OD₆₀₀>0.6. IPTG (isopropyl-β-D-thiogalactopyranoside) was added at final concentration of 0.1-1 mM, for induction of protein expression. Culture growth was continued for another 3 hr at 37° C., and 30° C. or for overnight at 16° C., according to predetermined optimization experiments. Cells were harvested by centrifugation (6000 rpm, 20 min, 4° C.).

Immediately before purification, cells were resuspended in 10-20 ml TBS (Tris-buffered saline—25 mM Tris-HCl, 137 mM NaCl, 2.7 mM KCl, pH 7.4), supplemented with 1 mM CaCl₂ (TBS-CaCl₂) and lysed by sonication. Sonication was performed on ice, using pulses to avoid overheating of the solution. The lysate was centrifuged (15000 rpm, 30 min, 4° C.), and the supernatant was used for protein purification.

His-Tagged Protein Purification

Purification using an FPLC AKTA-prime System (Amersham Pharmacia Biotech): The protein-containing supernatant was loaded on a 3-5 ml Ni-NTA column, pre-equilibrated with TBS-CaCl₂, at 0.5 ml/min. The column was washed with ˜40 ml TBS-CaCl₂ supplemented with 5 mM imidazole, 1 ml/min. Protein was eluted in TBS-CaCl₂ buffer and a linear gradient of 5-250 mM imidazole over ˜30 ml, 1 ml/min.

Fractions (1-2 ml) were collected and analyzed on SDS-PAGE (10-15%), and visualized by coomassie brilliant blue (CBB) staining. Fractions containing relatively pure protein were pooled and dialyzed overnight at 4° C. against TBS-CaCl₂.

CBM-Containing-Protein Purification

Batch purification: The supernatant was mixed with 10-15 ml of amorphous cellulose or five grams of Perloza MT 100 beaded cellulose (IONTOSORB, Usti nad Labem, Czech Republic), in a 50 ml tube, for one hr, on a rotator at 4° C. The amorphous/beaded cellulose was pelleted by centrifugation (4000 rpm, 5 min, 4° C.). The pellet was washed 3 times with ˜45 ml TBS, containing 1 M NaCl₂ and three times with 45 ml TBS. Each wash consisted of rapid vortexing (until the pellet was completely resuspended) and five min. rotation. Protein was eluted from the amorphous cellulose pellet by 12 ml (2-4 times) of 1% (v/v) triethylamine (TAE). Eluted fractions were quickly neutralized to pH˜7 with 0.5-1.5 ml of 1 M MES at pH 5.5 and dialyzed against TBS-CaCl₂. When beaded cellulose was used no further elution steps were applied. The beaded cellulose was supplemented with sodium azide (NaN₃) to a final concentration of 0.05% and stored at 4° C. Purity of either the elution fractions or the beaded cellulose was estimated by SDS-PAGE (10-15%).

Cohesin-Dockerin Based Affinity Chromatography

The supporting matrix, comprising 2 g of Perloza MT 100 beaded cellulose (IONTOSORB, Usti nad Labem, Czech Republic), suspended in 5 ml TBS, was packed into a C10-series column (GE Healthcare, Pittsburgh, Pa.). The column was then connected to an AKTA-Prime system (Amersham Pharmacia Biotech, Rehovot, Israel), and the flow rate was set at 1-3 ml/min throughout the experiment. The column was loaded with 54 μM of purified CBM-Coh, and then flushed with 30 ml of TBS-CaCl₂. A 5- to 20-ml cell extract of E. coli BL21 (λDE3) expressing the dockerin-tagged protein was applied to the column and washed with 30 ml of TBS-CaCl₂. The elution step was carried out under a gradient (0-250 mM) of EDTA in TBS, after which the system was equilibrated, for repeated applications, to its starting position with TBS-CaCl₂. The column flow-through and the elution fractions were collected and analyzed on SDS-PAGE.

When beaded cellulose on which CBM-Coh was directly purified was used, 1-2 ml were directly packed into a C10-series column. Except for the CBM-Coh loading step all of the following steps were the same as described above.

Protein Concentration and Preservation

Protein concentration was estimated by the absorbance at 280 nm and the extinction coefficient of the desired protein as calculated by Vector NTI program suite, (Invitrogen, Carlsbad, Calif.) from the known amino acid sequence. If needed, dilute protein solutions were concentrated using Vivaspin 2/6 ml, 5,000 MW cutoff—concentrators. Proteins were stored in 50% (v/v) glycerol at −20° C.

Truncated Dockerin-Containing Constructs

A construct coding for G. stearothermophilus xylanase T-6 with an EcoRI site at the 5′-terminus and a XhoI site at the 3′-terminus was produced using PCR (Handelsman et al., 2004). This construct was ligated at the EcoRI site with the PCR product of a C. thermocellum Cel48S dockerin gene (recombinant cellulosomal family-48 cellulase) (Wang et al., 1993), containing tandem 5′-terminal NcoI and BamHI sites and a 3′-terminal EcoRI site. The latter two PCR products were then inserted in concert into the pET-28a vector at the NcoI and XhoI sites. The resulting plasmid (pNDoc1) allows facile replacement of the Cel48S dockerin (termed hereafter Doc) with any other desired dockerin by digestion with EcoRI and either NcoI or BamHI. The resulting expressed product constitutes a C-terminal His-tagged xylanase T-6 fusion protein, bearing a dockerin at the N-terminus (termed Doc-Xyn). The desired truncated dockerins were generated by PCR, with a sense primer that introduced an NcoI site and an anti-sense primer that introduced an EcoRI site, utilizing wild-type (WT) Doc-Xyn as a template.

In order to challenge the performance of the truncated dockerin (Doc(Δ16)) as a purification tag, other candidates as diverse as possible were chosen, for example the jellyfish Aequorea victoria Green Fluorescent Protein (GFP), and the E. coli Maltose Binding Protein (MBP). In order to examine if the purification process has any effect on the enzyme activity, two enzymatically active proteins were chosen: the C. thermocellum β-glucosidase, and the E. coli Thioesterase/Protease I (TEP 1). In addition, two copies of the Staphylococcus aureus Fc binding B-domain of protein A (ZZ domain) were purified and additional aspects were applied to form a reusable antibody purification system.

Additional constructs were produced by replacement of Xyn with the above-mentioned enzymes, through digestion of the pET-28a vector with EcoRI and XhoI restriction enzymes and ligation of the respectively digested PCR products. The model proteins which contained the same restriction sites in the middle of their sequence were constructed through the Restriction Free (RF) method, as described above.

The resulting constructs are described and presented schematically in Table 1.

TABLE 1
Fusion protein constructs used in the affinity purification studies.

| | Molecular | Protein of | Affinity | Location of The | SEQ ID |
|---|---|---|---|---|---|
| Construct | Weight | Interest | Tag | Tag | NO: |
| wtDoc-Xyn | 53135 Da | Xyn | wtDoc | N-terminus | 123 |
| Doc(Δ16)- | 50785 Da | Xyn | ΔDoc | N-terminus | 124 |
| Xyn | | | | | |
| wtDoc-GFP | 35550 Da | GFP | wtDoc | N-terminus | 125 |
| Doc(Δ16)- | 33880 Da | GFP | ΔDoc | N-terminus | 126 |
| GFP | | | | | |
| GFP-wtDoc | 35270 Da | GFP | wtDoc | C-terminus | 127 |
| GFP- | 33600 Da | GFP | ΔDoc | C-terminus | 128 |
| Doc(Δ16) | | | | | |
| Doc(Δ16)- | 27670 Da | TEP1 | ΔDoc | N-terminus | 129 |
| TEP1 | | | | | |
| Doc(Δ16)- | 58472-Da | β-glucosidase | ΔDoc | N-terminus | 130 |
| BglA | | | | | |
| Doc(Δ16)-ZZ- | 22500 Da | ZZ-domain | ΔDoc | N-terminus | 131 |
| domain | | | | | |

All constructs had a C-terminal His tag except for the GFP-wtDoc and GFP-Doc(Δ16) constructs which included a His tag located at the N-terminus of GFP. It should be understood that the SEQ ID NOs listed in table 1 refer to the amino acid sequence of the corresponding constructs.

The full-length sequence of the dockerin-bearing protein encoded by the Cel48S dockerin, which contains a type I dockerin domain on its C-terminus, is set forth in SEQ ID NO: 7 (GenBank Accession #L06942). The wild-type Type-I dockerin domain, utilized in the Examples herein, is set forth in SEQ ID NO: 1. The truncated dockerin domain (Doc(Δ16)), utilized in the Examples herein, is set forth in SEQ ID NO: 6.

### CBM-Coh Construct

The gene encoding the protein construct (CBM-Coh), consisting of a carbohydrate-binding module (CBM) and a cohesin (Coh) from the C. thermocellum CipA (SEQ ID NO: 9; GenBank Accession #L08665), was cloned as previously described (Yaron et al., 1995).

TABLE 2
Primers used in cloning the constructs
of the invention.

| | | | SEQ ID |
|---|---|---|---|
| Construct | | Amino Acid sequence | NO: |
| Wt Doc | 5′ | CCCCATGGGATCCGGCGACGTCAATG | 132 |
| XynT6 | | ATGACGG | |
| | 3′ | GGGGAATTCGTTCTTGTACGGCAATG | 133 |
| | | TATC | |
| Wt/Doc | 5′ | GATGAGCAAGTTGGCCGJACAAGAAC | 134 |
| (Δ16)- | | GAATTCATGAGTACTCAGTGGTGGTG | |
| GFP | 3′ | GTGGTGGTGCTCGAGTTTGTAG | 135 |
| | | AGCTCATCCATGC | |
| GFP wtDoc | 5′ | ACCATGAGCCACCATCACCATCACCA | 136 |
| | | TATGAGTAAAGGAGAAGAACTT | |
| | 3′ | GTCATCATTGACGTCGCCAGGTACCA | 137 |
| | | CTTTGTAGAGCTCATCCATGCC | |
| Doc(Δ16)- | 5′ | CCATCAGAATTCATGGCGGACACGTT | 138 |
| TEP1 | | ATTGATTC | |
| | 3′ | CAGATACTCGAGTGAGTCATGATTTA | 139 |
| | | CTAAAG | |
| | 5′ | GTATCCGAATTCATGTCAAAGATAAC | 140 |
| | | TTTCCC | |
| | 3′ | GCATAACTCGAGAAAACCGTTGTTTT | 141 |
| | | TGATTAC | |
| Doc(Δ16)- | 5′ | GTACAAGAACGAATTCATGTCAAAGA | 142 |
| BglA | | TAACTTTCC | |
| | 3′ | GGTGGTGGTGCTCGAGAAAACCGTTG | 143 |
| | | TTTTTGATTAC | |
| Doc(Δ16) | 5′ | CACGGTGAATTCCTGGTGCCACGCGG | 144 |
| ZZ-domain | | TTCCATG | |
| | 3′ | CCAATGCTCGAGTGCAAGCTTGTCAT | 145 |
| | | CGTCGTC | |
| Doc(Δ16)- | 5′ | TATACCATGGGATCCAAGAGATATGT | 146 |
| XynT6 | | TTTGAGAT | |
| | 3′ | TCTTGAATTCGTTCTTGTACGGCAAT | 147 |
| | | GTATCTATTTCTTT | |

### ELISA-Based Affinity Assay

The ELISA-based cohesin-dockerin binding assay was performed essentially according to Barak et al (Barak et al., 2005) using a matching Coh-Doc fusion-protein system. The interaction of the test cohesin with the truncated dockerins was expressed as the function of change in Gibbs free energy (ΔΔG°), relative to the wild-type dockerin, calculated using equation 1:

ΔΔG=ΔG^{WT}−ΔG^{mut}=−RT ln(EC₅₀^{WT}/EC₅₀^{mut}) Equation 1:

where R is the gas constant, T the absolute temperature (° K), and the EC₅₀ was determined from the binding curves of the truncated Doc-Xyn fusion proteins, compared with that of the wild-type Doc-Xyn (Reichmann et al., 2007). To determine the EC₅₀ of the truncated dockerins, a nonlinear fit for the ELISA curves was calculated using the GraphPad Prism 4 program (GraphPad Software, Inc., La Jolla, Calif.).

In order to calculate the different K_{D} values (e.g., for Doc(Δ16)), ΔG^{WT} was first calculated by solving Equation 2 for K_{D}=1.7×10⁻¹⁰ (61) and T=298° K. Next, ΔG^{mut} was calculated by solving equation 1 with the previously calculated values of ΔΔG and ΔG^{WT}. Finally, the different K_{D} values were calculated by solving equation 2 once again with the calculated ΔG^{mut} value (and T=298° K).

ΔG=−RT ln(K_{D}) Equation 2:

Competitive ELISA

This competitive assay was done in order to measure the relative binding affinity of different fusion proteins to which no primary antibody were available. It was done similarly to the above-mentioned ELISA procedure with a few changes. ELISA plates were initially coated with CBM-cohesin (50 nM), blocked and washed as described. Next, different concentrations (1 pM-0.2 μM) of the dockerin-fused proteins (Doc(Δ16)-GFP, wtDoc-GFP) were mixed with a constant concentration of the wtDoc-Xyn (100 pM). This mixture was then added in duplicate into the wells for interaction. Subsequently, washing and detection steps were conducted as mentioned above. The wtDoc-Xyn interaction with the cohesin was challenged by increasing concentrations of the dockerin-fused test protein, which resulted in the reduction of recognition by the primary antibody and consequently in the signal produced by the secondary antibody.

To determine the inhibition concentration (IC₅₀) of the Doc(Δ16)/wtDoc, a nonlinear fit for the ELISA curves was calculated_{—} using one-site binding competitive equation (Equation 3) of the GraphPad Prism 4 program, using (GraphPad Software, Inc., La Jolla, Calif.). Log IC50 is the logarithm of the, IC50 (50% of the binding sites are occupied by the competitor).

$$Y = Bottom + \frac{( Top - Bottom )}{1 + {10}^{X - Log IC 50}} \qquad Equation 3$$

The changes in the Gibbs free energy (ΔΔG°) between the wild type and the truncated dockerin and their respective interactions with test cohesin were calculated using Equation 4.

ΔΔG=ΔG^{WT}−ΔG^{mut}=−RT ln(IC₅₀^{WT}/IC₅₀^{mut}) Equation 4:

### EXAMPLE 1

### Cohesin-Dockerin Affinity Purification Using Xylanase T-6

The aim of this study was to develop and optimize an efficient affinity-purification system, based on the CBM (carbohydrate-binding module) and cohesin-dockerin interaction. Beaded cellulose was used as the column support matrix for the immobilization of a type-I cohesin (Coh) module-containing CBM-Coh fusion protein, wherein the Coh module was from the same bacterium from which the cellulose was derived. This simple application step served as a non-covalent means for “activating” the column for subsequent purification of a matching dockerin-containing target protein. No leakage of the CBM-Coh fusion protein from the column was detected after extensive washing with buffer, and the column was then ready for protein purification.

The target protein destined for purification was fused to a truncated dockerin as an affinity tag and could be eluted effectively from the column by graded concentrations of EDTA. The regenerated cellulose:CBM-Coh column was available for subsequent use without significant reduction of its efficiency and capacity.

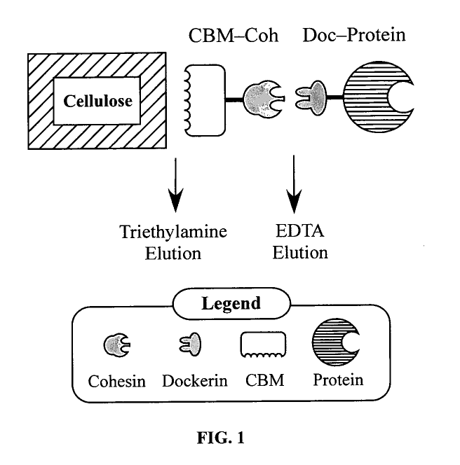

The first model target protein comprised G. stearothermophilus xylanase T-6, fused to a C. thermocellum dockerin (the affinity tag). A solution containing the dockerin-borne target protein was loaded onto the column, followed by extensive buffer washes in the presence of calcium. To elute the protein, a gradient of EDTA was applied, and protein elution was continuously monitored spectroscopically. The appropriate fractions were analyzed subsequently by SDS-PAGE. A schematic description of the approach is presented in FIG. 1.

### Cohesin-Dockerin Affinity Chromatography

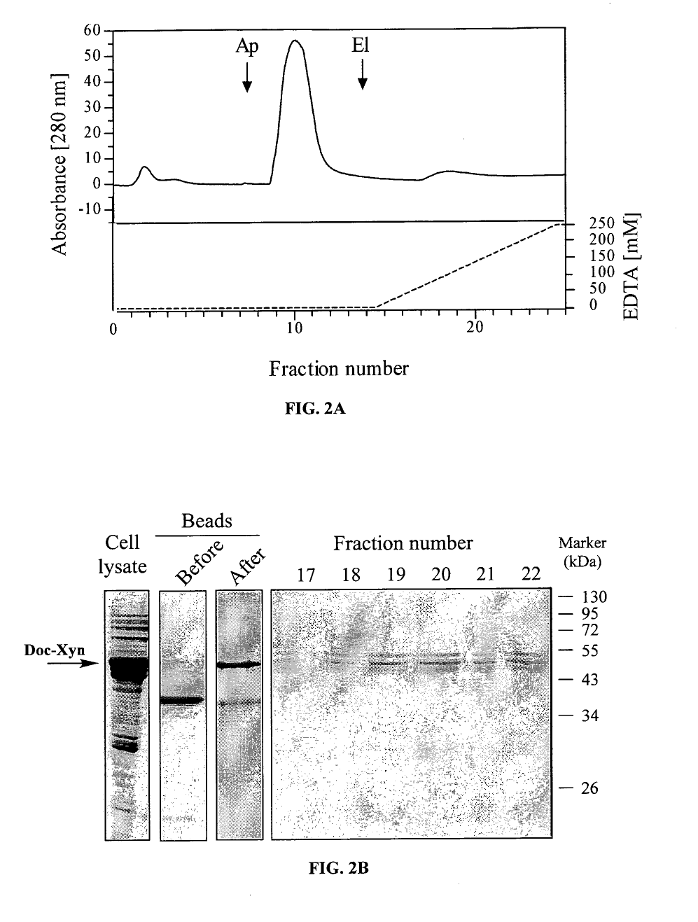

Cell lysates of E. coli, expressing the wild-type dockerin-xylanase chimaera (Doc-Xyn) were applied onto the activated column (FIG. 2A, Ap) resulting in a large peak corresponding to unbound protein which immediately passed through the column; the column was washed with TBS-Ca and then subjected to a gradient of EDTA to elute the bound protein (FIG. 2A, El). As seen from the chromatogram and the accompanying SDS-PAGE (FIG. 2B) of the fractions, very little protein was eluted from the column (FIG. 2B fractions 17-22). Most of the protein was retained on the beaded cellulose (FIG. 2B), suggesting that the cohesin-dockerin complex is too tight to dissociate using EDTA.

### EXAMPLE 2

### Truncation of Residues 2-17 of the Dockerin Domain Confers Reversible Binding to the Cohesin-Dockerin System and Reusability of the Affinity Column

To overcome the tight cohesion-dockerin association, a homologous series of truncated dockerins were created and used to replace the dockerin tag in Doc-Xyn. FIG. 3A depicts the sequences of the wild type dockerin and truncated derivatives. The two dockerin segments (conserved duplicated regions) are indicated on top. Residues involved in Ca²⁺ coordination are highlighted in gray. Black-highlighted (white font) residues represent those involved in direct hydrogen bonding to cohesin via the second duplicated repeat. Hydrogen-bonding residues in the alternative symmetry-related mode are shown in open boxes. Positions of the helices are marked h1, h2 and h3, respectively. The first and second calcium-binding loops are marked as Ca²⁺ loop-1 and Ca²⁺ loop-2, respectively. Binding affinities of the truncated dockerins to cohesin was assessed quantitatively by an ELISA-based method. Deletion of the N-terminal dockerin, from Asp² (residue 2 of SEQ ID NO:1; the first residue of the calcium-binding loop) up to Lys¹⁸ (residue 18 of SEQ ID NO:1) in the middle of the first α-helix, yielded Doc(Δ16), which retained binding at a level close to that of the wild-type module (ΔΔG=0.4 kcal/mol) (FIG. 3B-C). Further incremental expansion of the dockerin deletion served to almost entirely abolish its binding to cohesin.

Thus, Doc(Δ16) (also designated as ΔDoc) was examined as a short affinity tag for the purification of the target protein on the CBM-Coh-immobilized beaded cellulose. A total of 24 nmol of Doc(Δ16)-Xyn was recovered from the column after elution, close to the total amount loaded onto the column (27 nmol).

wtDoc- and Doc(Δ16)-Xyn were further evaluated for their binding to type-I cohesin not only in the presence of Ca²⁺ but also in the presence of EDTA (FIG. 3E). Cohesin-dockerin interactions were measured using the ELISA-based assay. 96-well plates (nunc™) were coated with CBM-Coh and interacted with either wtDoc-Xyn or Doc(Δ16)-Xyn in the presence of 1mM CaCl₂ or 10 mM EDTA. The wtDoc-Xyn presented the strongest binding in the presence of Ca²⁺, while in the presence of EDTA its binding was somewhat compromised (ΔΔG=2.01 kcal/mol). In the presence of Ca²⁺, Doc(Δ16)-Xyn interacted similarly to the wtDoc-Xyn supplemented with EDTA (ΔΔG=2.4 kcal/mol, between wild-type and truncated form supplemented with Ca²⁺), while in the presence of EDTA the Doc(Δ16)-Xyn failed to present any significant binding. These results demonstrate that the truncated DocS although lacking its first α-helix and the first Ca²⁺ binding loop retained relatively high binding capacities.

Furthermore, xylanase purified using the Doc(Δ16) in the cohesin-dockerin system exhibited enhanced purity compared to the purified by immobilized metal-ion affinity chromatography (IMAC) by virtue of the His tag (FIG. 3D). Thus, cohesin-dockerin affinity purification using Doc(Δ16) is superior to both cohesin-dockerin affinity purification using wild-type dockerin and to IMAC.

### EXAMPLE 3

### Cohesin-Dockerin Affinity Chromatography Columns Containing Doc(Δ16)-Xyn Exhibit Excellent Yield, Elution, and Reusability

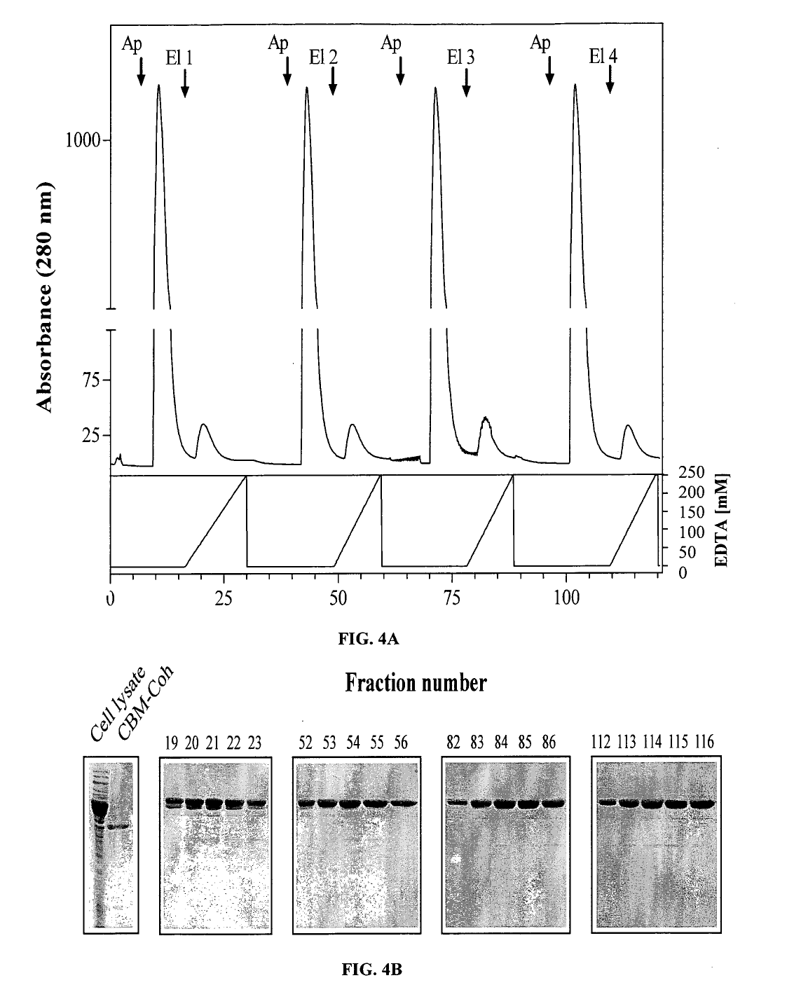

A critical feature for protein affinity purification is the ability to reuse the column several times without a decrease in performance. Four identical samples of the E. coli crude lysate (5 ml), containing the expressed Doc(Δ16)-Xyn, were applied (Ap) onto the column (2 ml of beaded cellulose) and eluted (El) using an EDTA gradient (FIG. 4A). The eluted fractions in each cycle were highly enriched with Doc(Δ16)-Xyn (FIG. 4B) without any apparent contaminating proteins or CBM-Coh. Nearly identical amounts of protein were purified in the successive rounds, underscoring the robustness of the affinity tag.

### EXAMPLE 4

### Cohesin-Dockerin Affinity Chromatography Columns Containing Doc(Δ16) Fused to the N-Terminus of the GFP Exhibit Excellent Yield, Elution, and Reusability

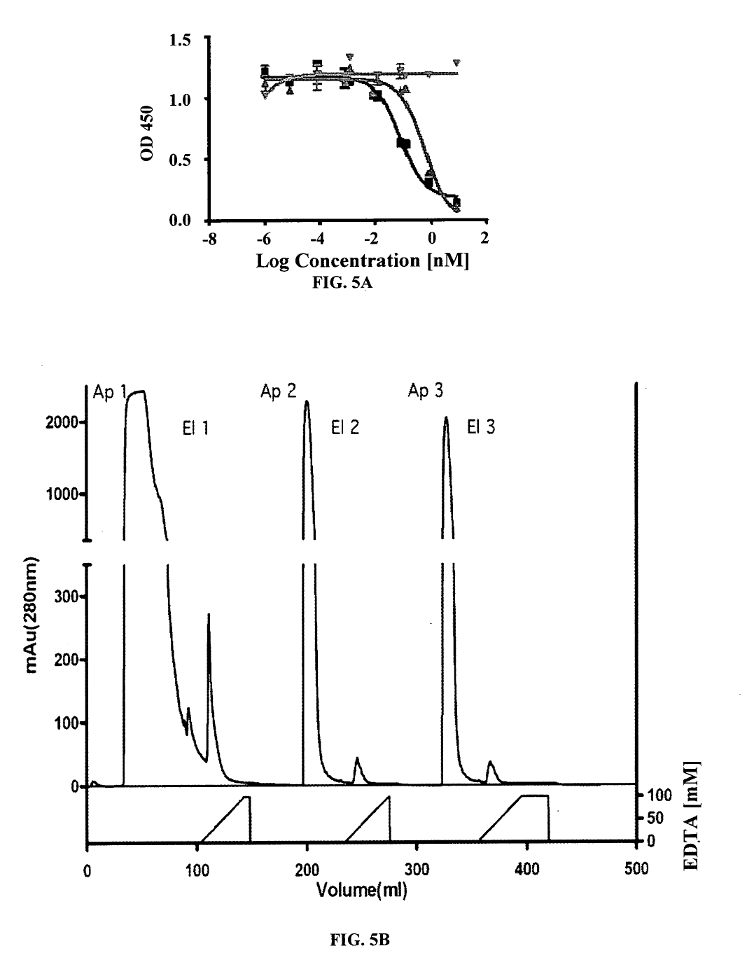

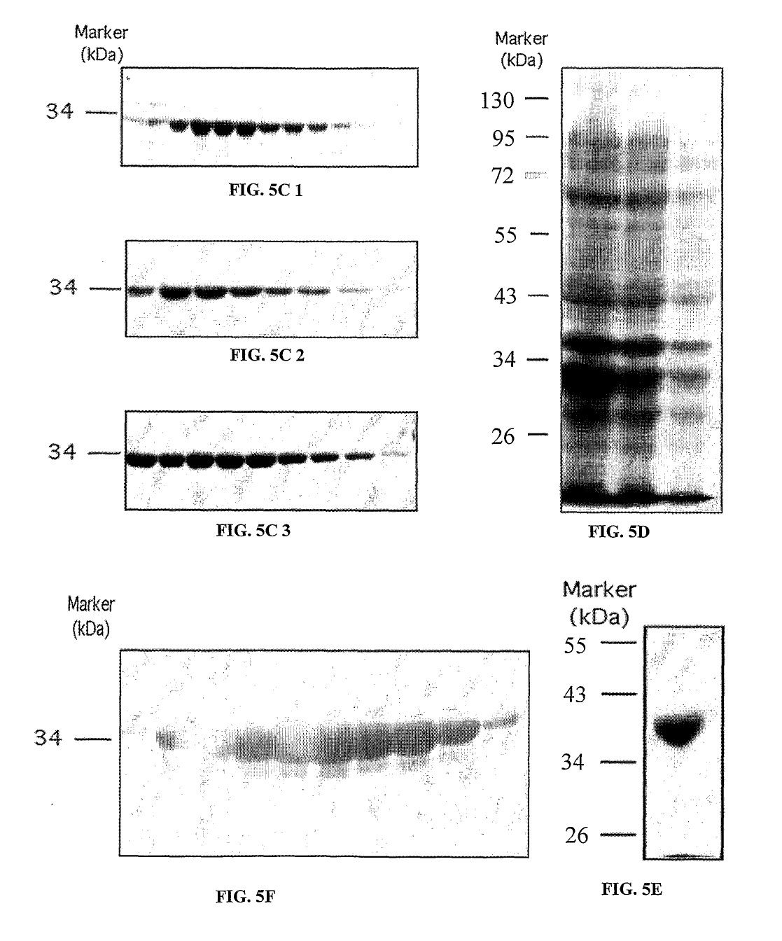

The binding capacity of a truncated dockerin GFP (Doc(Δ16)-GFP) was evaluated relatively to the wild type Doc GFP (wtDoc-GFP) by a competitive ELISA assay. Thus, an ELISA plate was coated with CBM-Coh and rising amounts of either wtDoc-GFP or Doc(Δ16)-GFP fusion proteins were mixed together with a constant amount of wtDoc-Xyn. The reaction was done in the presence of 1 mM CaCl₂. After appropriate incubation time the plate was washed and examined with anti-Xyn antibodies. As can be seen (FIG. 5A), the truncated dockerin had similar affinity to that of the wild-type module (ΔΔG=1.2 kcal/mol). The competitive ELISA adds another parameter to the system thus making it more accurate. This experiment indicates that the Xyn fusion had but a minor effect on the interaction between the dockerin and the cohesin and further substantiates the dual-binding mode of the dockerin module.

In order to evaluate column durability, cell lysates of E. coli expressing Doc(Δ16) fused to the N-terminus of the GFP were applied and eluted consecutively using the immobilized cohesin column. Samples of the E. coli crude lysate (20 ml), containing the expressed Doc(Δ16)-GFP, were applied (Ap) onto the column (2 ml of beaded cellulose 2 mg CBM-Coh) and eluted (El) using an EDTA gradient. Following the first elution, two consecutive applications of the unbound protein (˜5-10 ml) were further applied and eluted. The different stages were monitored throughout the procedure by following the absorbance at 280 nm (FIG. 5B). The eluted fractions were analyzed subsequently by SDS-PAGE (˜40-50 nmol purified protein per elution) (FIG. 5C 1-3).

The first elution (EL 1; FIG. 5B) indicates that the protein band corresponding to the calculated size of the Doc(Δ16)-GFP appears to be homogeneous and enriched after only one purification step. Thus, the washing step was insufficient and the first elution commenced before the entire unbound fraction was released, corresponding to the relatively high absorbance peak on the chromatogram, and to the minor impurities on the gel. In the following applications, the washing step was extended for longer periods of time, which resulted in relatively smaller absorbance peaks but highly homogenous protein bands.

Since the column is based on the direct binding between the cohesin and the Doc(Δ16), the maximum amount of obtained protein corresponds to the relatively small amount of pre-incubated CBM-Coh. Thus, it is not surprising to notice large amounts of proteins in the flow-through fractions (FIG. 5D). The protein found in the flow through was properly folded and bound to the column in the consecutive application as can be observed in the reduction in the amount of protein found in the unbound fractions. Each consecutive elution, not only retained the column capacity, but also achieved better results stemming from increased washing period. Even in the third elution single band in highly purified state is observed, serving as evidence to the robustness of the system (FIG. 5C 3).

In order to test the effectiveness of the elution step, some column beads were removed and subjected to SDS-PAGE after the final application was eluted and the column was extensively washed. The beads appear to contain only a single band corresponding to the molecular weight of the CBM-Coh, thus indicating that the Doc(Δ16)-GFP was completely eluted following the application of EDTA (FIG. 5E).

In order to compare the new affinity system to the commonly used IMAC, Doc(Δ16)-GFP was purified through its His tag rather than the ΔDoc affinity tag (FIG. 5F). Although high amounts of protein were received, a very close band was seen adjacent to the eluted protein. This band was absent from any of the elutions observed when using the Doc(Δ16) system, demonstrating its supremacy and its high specificity in protein purification. In order to examine whether this contaminating band may represent cleavage between the Doc(Δ16) and the GFP, a highly purified sample was sent to N-terminal sequencing. While the upper band was recognized as the beginning of the Doc(Δ16) the lower band could not be identified. This may imply that the truncation occurred in several close positions and not in one specific position or that it is an unrelated contamination of the host proteins with exposed histidine residues that were captured on the Ni beads.

### EXAMPLE 5

### Cohesin-Dockerin Affinity Chromatography Columns Wherein Wild-Type Dockerin is Fused to the N-Terminus of GFP

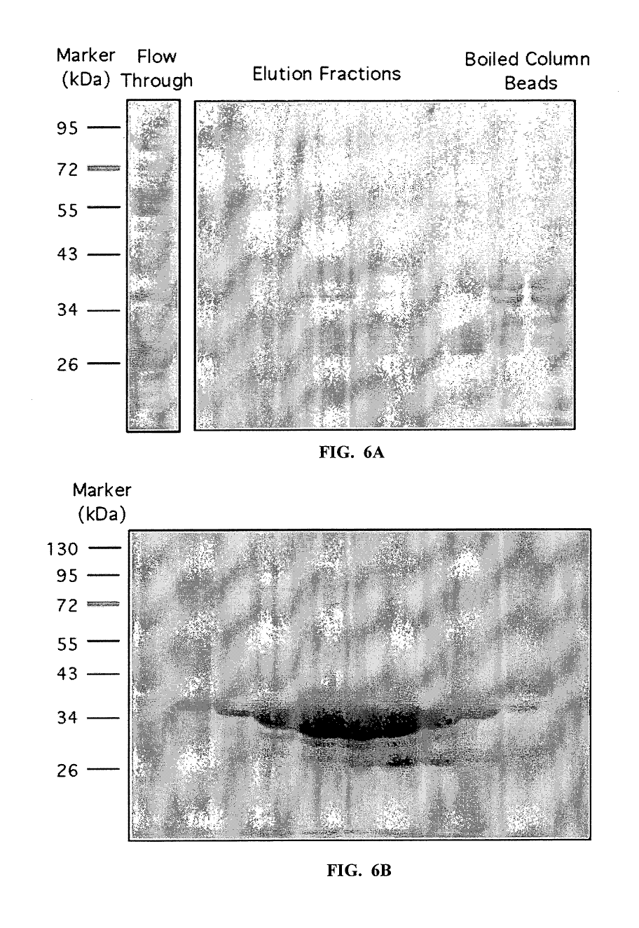

Unlike the affinity system described by Craig et al., GFP with wild-type dockerin at the N-terminus (wtDoc-GFP) binding to the CBM-Coh was too strong and impossible to elute the fusion protein even with concentrations as high as 500 mM EDTA (FIG. 6A). It can be seen that the wtDoc-GFP retained on the column bound to the CBM-Coh and was separated after boiling the column beads (Perloza beaded cellulose) with SDS for 5 mM. Only negligible amounts of wtDoc-GFP could be seen in the elution fraction. The CBM-Coh has a higher molecular weight (˜36 kDa), therefore it probably represents the upper band in the boiled beads fraction, whereas the wtDoc-GFP is the lower one.

In order to rule out the possibility that the wtDoc-GFP did not express well in the bacteria, a C-terminal His tag allowed an alternative affinity system (Ni-NTA) for purification of the protein (FIG. 6B). By applying increasing amounts of imidazole, large amounts of protein was obtained from the bacteria cell lysate, indicating that the strong binding of the intact dockerin, rather than any expression difficulties, was the reason for the poor protein elution using the CBM-Coh column.

### EXAMPLE 6

### Cohesin-Dockerin Affinity Chromatography Columns Containing Doc(Δ16) Fused to the C-Terminus of the GFP Exhibit Excellent Yield, Elution, and Reusability

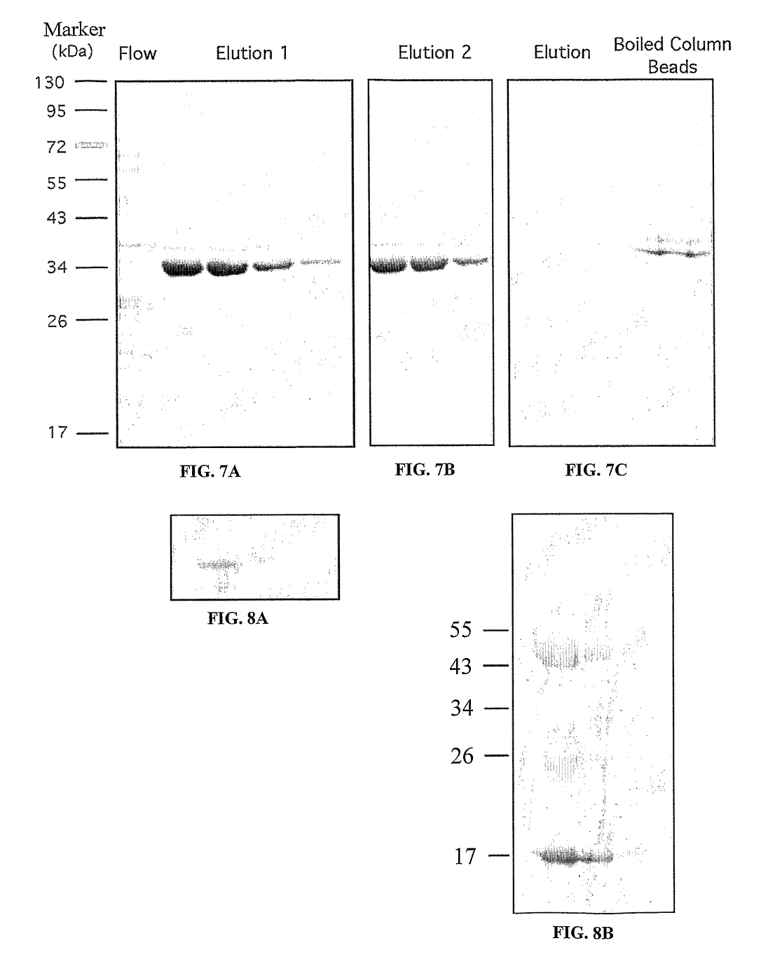

A good affinity tag should exhibit similar qualities (specific attachment and efficient elution) when fused either to the N or the C-terminus of the protein of interest, in order to extend the purification options. However, the same affinity tag may have a very different effect on the expression, solubility and/or stability properties of a protein when fused to its N- rather than its C-terminus or vice versa. Thus, two additional versions of the GFP dockerin fusion proteins were constructed, wherein the truncated DocS (Doc(Δ16)) or the wild type DocS (wtDoc) was positioned at the C-terminus of the GFP, and a His tag was positioned at the N-terminus. Under similar conditions as previously described, a relatively pure band corresponding in size to GFP-Doc(Δ16) was specifically purified in two consecutive applications of cell lysate (FIG. 7A-B), demonstrating the ability of the tag to be fused and remain active at the C-terminus of the protein.

Nevertheless, unlike in the previous section where no contaminations were seen in the CBM-Coh purified protein, an additional band appeared. This may imply that some cleavage events may have occurred at the N-terminus of the GFP-Doc(Δ16) during the expression in the bacterial host, while the C-terminally positioned Doc(Δ16) retained the ability to interact with the CBM-Coh. Alternatively, bond breakage may have occurred after the purification (during dialysis or storage). On the other hand, this band may still represent some contamination.

Similar to the wtDoc-GFP the GFP-wtDoc did not elute from the column and could be seen together with CBM-Coh after boiling the column beads in the presence of SDS (FIG. 7C).

### EXAMPLE 7

### Cohesin-Dockerin Affinity Chromatography Columns Containing Doc(Δ16) Fused to the N-Terminus of the ZZ Domain

The ZZ-domain is a synthetic analogue of the B-domain of protein A from the bacteria Staphylococcus aureus. It is widely used in research and in the industry due to its ability to bind the heavy chain Fc region of immunoglobulins. Often the ZZ-domain is immobilized onto a solid support and used as a purification method of total IgG from blood serum. Attaching a detachable affinity tag to the ZZ-domain could make it reusable and more applicable not only for antibody purification but for other nanotechnological applications.

Therefore, Doc(Δ16) fused at the N-terminus of the ZZ-domain was cloned and expressed (FIG. 8A). Due to its repetitive nature, ZZ-domain was cloned with an addition of 20 amino acids (7 upstream and 13 downstream), which enables the cloning of the two identical domains together. The resulting protein (Doc(Δ16)-ZZ) was immobilized onto beaded cellulose through CBM-Coh interaction. Subsequently, diluted mouse serum was applied onto the column, and, after extensive washes with TBS-CaCl₂, IgG's were eluted using glycine buffer (0.1M pH 2.8). The Doc(Δ16)-ZZ specifically bound antibodies from mouse total blood serum (FIG. 8B) as can be deduced from molecular weight of the eluted proteins (50 kDa and 25 kDa) corresponding to the 150 kDa molecular weight of IgG's heavy and light chains.

### EXAMPLE 8

### Purification and Activity Assay of the BglA Fusion Protein

Many affinity tags usually have some influence on their adjacent proteins, and therefore have to be removed. On the other hand, the His tag is considered to have a relatively minor effect on the protein when added, and is therefore a preferred affinity tag in this respect. Therefore, the C. thermocellum β-glucosidase (BglA) was purified, and the effect of the Doc(Δ16) tag on its fused protein function was examined.

The C. thermocellum β-glucosidase has a crucial role in the degradation of cellulose. It hydrolyzes the cellobiose—a strong inhibitor of the cellulase system, into fermentable glucose thus allowing the degradation of cellulose to continuously proceed. Enhancement of cellulose degradation was previously observed when free β-glucosidases from different sources were supplemented to cellulose-degrading enzymes. In addition, the thermophilic origin of the β-glucosidase makes it potentially useful in industrial saccharification of cellulosic substances even as the free enzyme, i.e., not supplemented to a cellulose-degrading system. Therefore, BglA was chosen as a model for enzymatic studies.

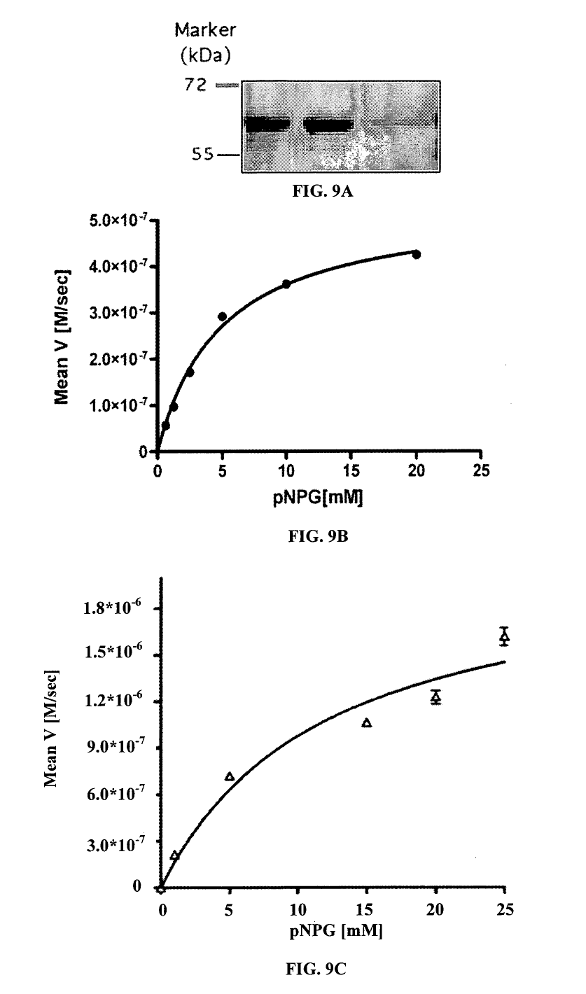

Truncated DocS was fused to the N-terminus of β-glucosidase (Table 1, Doc(Δ16)-BglA), expressed and purified on CBM-Coh bound to beaded cellulose. A single band of about ˜58 kDa, corresponding to the calculated size of the fusion protein, can be seen (FIG. 9A). Two consecutive elutions were performed each produced a relatively pure single major band. The addition of the Doc(Δ16) tag had no significant effect on the activity of the enzyme compared to the activity of the wild-type enzyme (without the dockerin module, containing only a His tag; FIG. 9C), which was purified using a Ni-NTA affinity column. ΔDoc-BglA had kinetic parameters on the same order of magnitude as the wild-type BglA (with His tag) with slightly better values (Table 3) (FIG. 9B). The enzyme activity was tested at 50° C., similar to the environmental conditions of the thermophilic bacterium and temperature optimum of the enzyme. The curves were fitted using the Graphpad prism program.

TABLE 3
Kinetic parameters of different BglA dockerin derivatives

| | Vₘₐₓ [M s⁻¹] | Kₘ [mM] | k_{cat} [s⁻¹] | k_{cat}/Kₘ [mM⁻¹ s⁻¹] |
|---|---|---|---|---|
| Wt BglA | 2.2E−06 | 12.08 | 21.53 | 1.8 |
| ΔDoc BglA | 5.4E−07 | 4.9 | 53.8 | 10.9 |
| BglA Doc Cc | 7.7E−07 | 13.02 | 7.7 | 0.6 |

The addition of the free β-glucosidase to cellulosomal systems was proved to enhance their degradation activities by removing the inhibitory properties of the cellobiose produced by converting the disaccharide to the non-inhibitory glucose. In light of this, it is hypothesized that the addition of dockerin-fused β-glucosidase could enhance the degradation process even more than the addition of the free enzyme by creating a proximity effect.

### EXAMPLE 8

### Purification and Activity Assay of the TEP1 Fusion Protein

E. coli thioesterase I (TEP1) has been documented to execute diverse activities of thioesterase, esterase, arylesterase, protease, and lysophospholipase. TEP1 was first shown to catalyze hydrolytic cleavage of acyl-CoA thioesters. Subsequently, the same enzyme was isolated using different types of substrates and thus received alternative names, i.e., protease I for hydrolyzing N-acetyl-DL-phenylalanine-2-naphthyl ester, and lysophospholipase L₁, for hydrolysing the 1-acyl group of lysophospholipid. Though the physiological role of TEP 1 is unclear, it has been suggested to be potentially useful for the kinetic resolution of racemic mixtures of industrial chemicals.

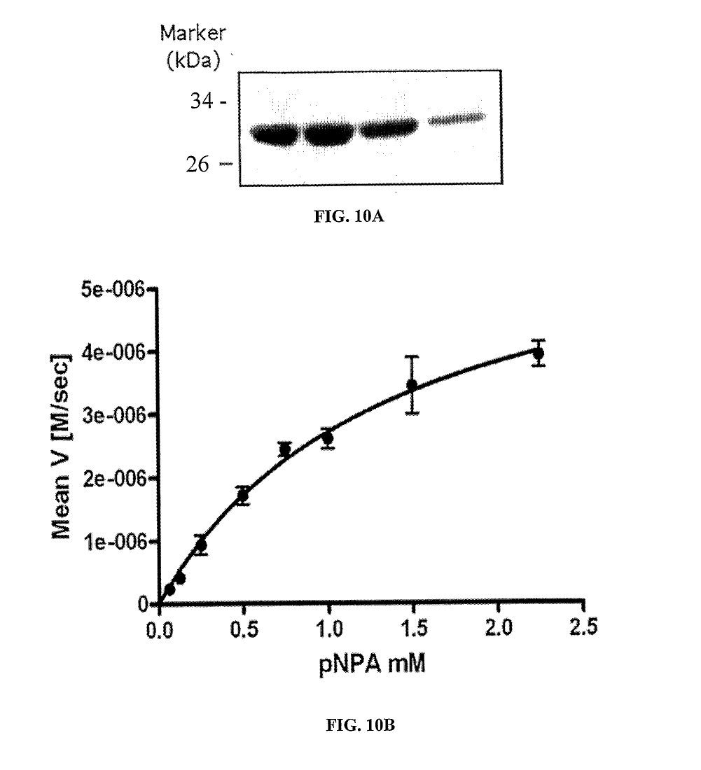

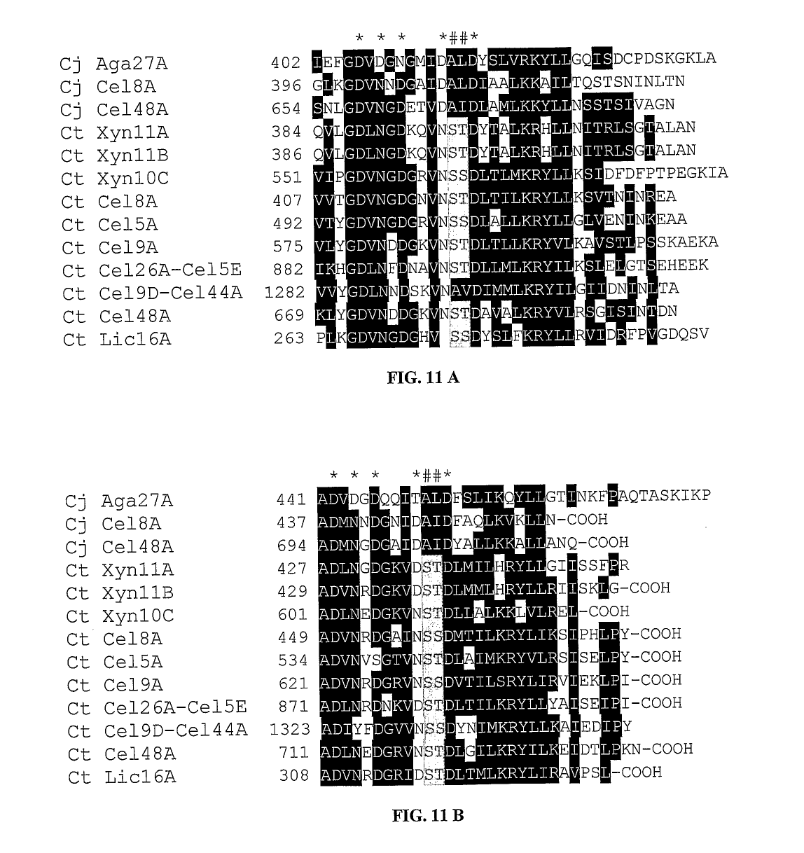

From the wide variety of catalyzed reactions, the esterase ability of the TEP1 was examined, using the readily available substrate 4-nitrophenyl acetate (pNPA). Truncated DocS was fused to the N-terminus of TEP1 (Doc(Δ16)-TEP1), expressed and purified on a CBM-Coh bound to beaded cellulose. A single band of about ˜27 kDa, corresponding to the calculated size of the fusion protein, was observed in SDS-PAGE gels (FIG. 10A). Two consecutive elutions were performed showing a similar single band displaying the high robustness of the purification system. The enzyme was active with a Vmax of 6.42 E-06 M s⁻¹ on pNPA and Km of 1.098 mM (FIG. 10B, curve fitted using the Graphpad prism program; table 4).

TABLE 4
Kinetic parameters of the Doc(Δ16)-TEP1.

| | Vₘₐₓ [M s⁻¹] | Kₘ [mM] | k_{cat} [s⁻¹] | k_{cat}/Kₘ [mM⁻¹ s⁻¹] |
|---|---|---|---|---|
| ΔDoc-TEP1 | 6.42E−06 | 1.37 | 12.83 | 9.37 |

The foregoing description of the specific embodiments will so fully reveal the general nature of the invention that others can, by applying current knowledge, readily modify and/or adapt for various applications such specific embodiments without undue experimentation and without departing from the generic concept, and, therefore, such adaptations and modifications should and are intended to be comprehended within the meaning and range of equivalents of the disclosed embodiments. Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

### REFERENCES

Barak, Y, Handelsman, T, Nakar, D, Mechaly, A, Lamed, R, Shoham, Y, Bayer, E A. 2005. J Mol. Recogit. 18: 491-501.
Bayer, E A, Chanzy, H, Lamed, R, Shoham, Y. 1998. Curr. Opin. Struct. Biol. 8: 548-557.
Bayer, E A, Shimon, L J W, Lamed, R, Shoham, Y. 1998. J. Struct. Biol. 124: 221-234.
Bayer E A et al., FEBS Lett. 1999; 463:277-80
Bayer, E A, Belaich, J-P, Shoham, Y, Lamed, R. 2004. Annu. Rev. Microbiol. 58: 521-554.
Craig, S J, Foong, F C, Nordon, R. 2006. J Biotechnol 121: 165-173.
Ding S-Y, Bayer E A, Steiner D, Shoham Y, Lamed R. 1999. J. Bacteriol. 181: 6720-6729.
Ding S-Y, Bayer E A, Steiner D, Shoham Y, Lamed R. 2000. 182: 4915-4925.
Doi R H, Goldstein M, Hashida S, Park J S, Takagi M. Crit Rev Microbiol. 1994; 20(2):87-93
Fierobe H P, Pagès S, Bélaïch A, Champ S, Lexa D, Bélaïch J P. 1999 Sep. 28; 38(39):12822-32
Gerngross U T, Romaniec M P, Kobayashi T, Huskisson N S, Demain A L. Mol Microbiol. 1993 April; 8(2):325-34. Erratum in: Mol Microbiol 1993 December; 10(5):1155.
Gold and Martin. J Bacteriol. 2007; 189(19):6787-95.
Haimovitz, R, Barak, Y, Morag, E, Voronov-Goldman, M, Lamed, R, Bayer, E A. 2008. Proteomics 8: 968-979.
Handelsman, T, Barak, Y, Nakar, D, Mechaly, A, Lamed, R, Shoham, Y, Bayer, E A. 2004. FEBS Lett. 572: 195-200.
Jindou, S, Kajino, T, Inagaki, M, Karita, S, Beguin, P, Kimura, T, Sakka, K, Ohmiya, K. 2004. Biosci. Biotechnol. Biochem. 68: 924-926.
Kakiuchi M, Isui A, Suzuki K, Fujino T, Fujino E, Kimura T, Karita S, Sakka K, Ohmiya K. J Bacteriol. 1998, 180(16):4303-8
Karpol, A, Barak, Y, Lamed, R, Shoham, Y, Bayer, E A. 2008. Biochem. J. 410: 331-338.
Kirby J, Martin J C, Daniel A S, Flint H J. 1997. FEMS Microbiol. Lett. 149: 213-219.
Lamed R, Setter E, Kenig R, Bayer E A. 1983. Biotechnol. Bioeng. Symp. 13: 163-181.
Lamed R, Naimark J, Morgenstern E, Bayer E A. J Bacteriol 1987, 169(8):3792-800
Mechaly, A, Yaron, S, Lamed, R, Fierobe, H-P, Belaich, A, Belaich, J-P, Shoham, Y, Bayer, E A. 2000. Proteins 39: 170-177.
Mechaly A, Fierobe H P, Belaich A, Belaich JP, Lamed R, Shoham Y, Bayer E A. J Biol Chem. 2001 Mar. 30; 276(13):9883-8. Erratum in: J Biol Chem 2001 Jun. 1; 276(22):19678.
Nakar, D, Handelsman, T, Shoham, Y, Fierobe, H-P, Belaich, J P, Morag, E, Lamed, R, Bayer, E A. 2004. J. Biol. Chem. 279: 42881-42888.
Ohara H, Karita S, Kimura T, Sakka K, Ohmiya K. Biosci Biotechnol Biochem. 2000, 64(2):254-60
Pagès, S, Belaich, A, Belaich, J-P, Morag, E, Lamed, R, Shoham, Y, Bayer, E A. 1997. Proteins 29: 517-527.
Pagès S, Belaich A, Tardif C, Reverbel-Leroy C, Gaudin C, Belaich J P. 1996. J Bacteriol 178: 2279-86
Pohlschröder M, Leschine S B, Canale-Parola E. J Bacteriol. 1994 January; 176(1):70-6.
Reichmann, D, Rahat, O, Cohen, M, Neuvirth, H, Schreiber, G. 2007. Curr. Opin. Struct. Biol. 17: 67-76.
Shoseyov O, Takagi M, Goldstein M A, Doi R H. Proc Natl Acad Sci USA. 1992 Apr. 15; 89(8):3483-7.
Van den Ent, F., and Lowe, J., 2006, J Biochem Biophys Methods 67, 67-74.
Wang, W K, Kruus, K, Wu, J H D. 1993. J Bacteriol. 175: 1293-1302.
Wu W Y et al., Nat Protoc. 2006; 1(5):2257-62.
Yaron, S, Morag, E, Bayer, E A, Lamed, R, Shoham, Y. 1995. FEBS Lett. 360: 121-124.
Zverlov V V et al., Proteomics. 2005; 5:3646-53.

1.-57. (canceled)

58. An affinity purification system, comprising a solid substrate, a bound protein comprising a cohesin domain, a recombinant or synthetic polypeptide comprising a molecule of interest and a truncated dockerin polypeptide derived from a dockerin domain, wherein the truncated dockerin polypeptide comprises only one calcium binding motif.

59. The affinity purification system of claim 58, wherein the solid substrate comprises cellulose and said protein bound to said solid substrate further comprises a carbohydrate-binding module (CBM), and wherein said solid substrate is selected from the group consisting of a bead, a cell, an extracellular matrix, and a container.

60. The affinity purification system of claim 58, wherein said dockerin domain is a Type I dockerin domain and said cohesin domain is a Type I cohesin domain.

61. The affinity purification system of claim 58, wherein said molecule of interest is a molecule other than a protein.

62. The affinity purification system of claim 58, wherein said molecule of interest is selected from the group consisting of a peptide, an enzyme other than xylanase, a peptide hormone, an antibody, or an antibody-binding domain wherein said affinity purification system further comprises at least the antigen binding portion of an antibody bound to said antibody-binding domain.

63. The affinity purification system of claim 58, wherein said truncated dockerin polypeptide contains a 14-16 amino acid deletion N-terminal to the lysine residue at position 18 of the wild-type Type I dockerin domain sequence.

64. A recombinant or synthetic polypeptide comprising a molecule of interest and a truncated dockerin polypeptide derived from a dockerin protein domain, wherein the truncated dockerin polypeptide comprises only one calcium binding motif.

65. The recombinant or synthetic polypeptide of claim 64, wherein said molecule of interest is a molecule other than a protein.

66. The recombinant or synthetic polypeptide of claim 64, wherein said molecule of interest is selected from the group consisting of a peptide, an enzyme, a peptide hormone and an antibody.

67. The recombinant or synthetic polypeptide of claim 64, wherein said truncated dockerin polypeptide contains a 14-16 amino acid deletion N-terminal to the lysine residue at position 18 of the wild-type Type I dockerin domain sequence.

68. The recombinant or synthetic polypeptide of claim 64, said truncated dockerin polypeptide further comprises an N-terminal glycine residue, wherein the glycine residue is attached directly to the truncated dockerin polypeptide.

69. The recombinant or synthetic polypeptide of claim 64, wherein the dockerin domain is a Type-I dockerin protein domain, or wherein the Type-I dockerin domain has the amino acid sequence as set forth in any one of SEQ ID NO:1, SEQ ID NO:36-SEQ ID NO:122, or an analog or derivative thereof.

70. The recombinant or synthetic polypeptide of claim 64, wherein the calcium binding motif is in the first segment of the dockerin domain or wherein the calcium binding motif is in the second segment of the dockerin domain.

71. The recombinant or synthetic polypeptide of claim 64, wherein the truncated dockerin polypeptide comprises the amino acid sequence as set forth in any one of SEQ ID NO: 2-SEQ ID NO: 4 or an analog or fragment thereof.

72. The recombinant or synthetic polypeptide of claim 66, wherein said molecule of interest is a peptide, the truncated dockerin polypeptide is linked to the N-terminus of said peptide or wherein the truncated dockerin polypeptide is linked to the C-terminus of said peptide.

73. A method of attaching a molecule of interest to a solid substrate, the method comprising the step of:
a) providing a solid substrate associated with a protein comprising a cohesin domain;
b) providing a molecule of interest covalently bound to a truncated dockerin polypeptide, wherein the truncated dockerin polypeptide comprises only one calcium binding motif;
c) allowing the truncated dockerin molecule to bind to the cohesin domain;
thereby attaching a molecule of interest to a solid substrate.

74. A method of purifying a molecule of interest, the method comprising the steps (a), (b), and (c) of claim 73, the method further comprises step d) eluting the molecule of interest of (b); thereby purifying a molecule of interest.

75. The method of claim 74, wherein said cohesin domain is a Type I cohesin domain, and said dockerin domain is a Type I dockerin domain.

76. The method of claim 74, wherein the solid substrate comprises cellulose and said protein associated with said solid substrate further comprises a carbohydrate-binding module (CBM), and wherein said solid substrate is selected from the group consisting of a bead, a cell, an extracellular matrix and a container.

77. The method of claim 74, wherein the step of attaching a truncated dockerin molecule to the cohesin domain is performed in the presence of Ca²⁺.

78. The method of claim 74, wherein the step of eluting the molecule of interest is performed with a chelator of a divalent cation.

79. A method of purifying a molecule of interest, said method comprising the steps of contacting a solid substrate with the molecule of interest and eluting said molecule of interest, wherein said solid substrate comprises:
a) a protein bound thereto comprising a cohesin domain;
b) an antibody-binding domain covalently bound to a truncated dockerin polypeptide comprising only one calcium binding motif; and
c) at least the antigen binding portion of an antibody bound to the antibody-binding domain, wherein the antibody recognizes said molecule of interest.

80. An isolated truncated dockerin polypeptide derived from a dockerin domain, wherein the truncated dockerin polypeptide comprises only one calcium binding motif and an analog, derivative and fragment thereof.

81. The truncated dockerin polypeptide of claim 80, wherein the dockerin domain is a Type-I dockerin protein domain, or wherein the Type-I dockerin domain comprises the amino acid sequence as set forth in any one of SEQ ID NO:1, SEQ ID NO:36-SEQ ID NO:122, or an analog or derivative thereof.

82. The truncated dockerin polypeptide of claim 80, wherein the analog comprises at least 70% homology to SEQ ID NO:1.

83. The truncated dockerin polypeptide of claim 80, wherein the calcium binding motif is in the first segment of the dockerin domain, or wherein the calcium binding motif is in the second segment of the dockerin domain.

84. The truncated dockerin polypeptide of claim 80, wherein the dockerin domain is from a thermophilic microorganism, or wherein the thermophilic microorganism is selected from a group consisting of Clostridium thermocellum and Archaeoglobus fulgidus.

85. The truncated dockerin polypeptide of claim 80 comprising the amino acid sequence as set forth in SEQ ID NO:4, or an analog or fragment thereof.

86. The truncated dockerin polypeptide of claim 80 comprising the amino acid sequence as set forth in any one of SEQ ID NO: 2-SEQ ID NO: 3, or an analog or fragment thereof.
